(19) United States
(12) Patent Application Publication
(10) Pub. No.: US 2012/0009602 A1
(43) Pub. Date: Jan. 12, 2012

(54) Reagents and Methods for Assessment of Human Tumor Malignancy and for Targeting Malignant Tumor Cells

(76) Inventors: Shib Das BANERJEE, Mysore (IN); Rajeev K. Boregowda, Hersey, PA (US)

(21) Appl. No.: 12/830,969

(22) Filed: Jul. 6, 2010

(51) Int. Cl. G01N 33/574; C12N 7/00; G01N 33/53; C07K 16/18

(52) U.S. Cl. 435/7.23; 530/388.1; 530/391.3; 530/391.7; 435/235.1; 435/7.21

(57) ABSTRACT

The disclosure relates to a monoclonal antibody, designated MAbH11B2C2, that binds specifically with a human hyaluronan-binding protein, designated H11B2C2. This protein can occur in two forms, one (apparently expressed by all malignant cells) having an apparent molecular size (as assessed by SDS-PAGE) of about 57 KDa, and another having an apparent molecular size of about 37 KDa. H11B2C2 is expressed by all malignant cell types tested, but does not appear to be significantly expressed by non-tumor cells. H11B2C2 can be used as a marker of malignant status, and MAbH11B2C2 can be used as a reagent for detecting malignant cells, for delivering agents to malignant cells, or both.

### BACKGROUND OF THE DISCLOSURE

The disclosure relates generally to the field of oncology, particularly to reagents that specifically recognize and bind with tumor tissues and cells.

The disclosure can be better understood against the background of present knowledge regarding extracellular matrix materials (especially hyaluronic acid and hyaluronan binding protein) and the significance of interactions among cells (especially tumor cells) and extracellular matrices. A relevant portion of this background subject matter is described in this section.

Extracellular Matrix and Hyaluronic Acid

Most animal cells are in contact with an intricate meshwork of interconnected extracellular macromolecules that constitute the extracellular matrix (ECM). Fibroblasts and other cells situated within or adjacent the ECM secrete the macromolecules that constitute the ECM. In specialized ECM structures, such as cartilage and bone, these macromolecules are secreted locally by specialized cells, such as chondroblasts in cartilage and osteoblasts in bone.

Two of the main classes of extracellular macromolecules that make up the matrix are polypeptide collagens and polysaccharide glycosaminoglycans (GAGs). GAGs are often covalently linked to proteins, these complexes being designated proteoglycans (PGs). GAGs and PGs form a highly hydrated, gel-like “ground substance” in which collagen fibers are embedded. The collagen fibers strengthen and help to organize the ECM. The aqueous phase of the polysaccharide gel permits diffusion of nutrients, metabolites, hormones, and other relatively small molecules among blood and tissue cells. In many cases, fibers of flexible proteins, designated elastins, are also present and impart resilience to the ECM. Two high molecular weight glycoproteins, fibronectin and laminin, are also common components of ECM.

ECM has a significant physiological role in tissue architecture, and also significantly affects cell behavior and cell differentiation. Many of the effects of ECM macromolecules are mediated by cell surface binding sites or receptors. Among ECM elements, hyaluronic acid (HA; also designated hyaluronan) exhibits a variety of roles and functions.

HA is a high molecular weight, linear GAG that occurs widely throughout the body, for example in body fluids, ECM, connective tissues, and stromal cells. HA is a polymer composed of a repeating disaccharide unit of glucuronic acid and N-acetylglucosamine (i.e., a polymer having the repeating subunit -beta(1,4)-D-glucuronic acid-beta(1,3)-D-N-acetylglucosamine-). Unlike other GAGs, HA contains no sulfated groups. HA is synthesized at the plasma membrane rather than in the Golgi complex. HA is believed to be elongated by addition of saccharide units at its reducing terminus, rather than at its non-reducing terminus. HA molecules are huge—not only in terms of their molecular weight, but also in terms of the space the molecules occupy in solution. HA appears to have one or more significant roles in regulating cell behavior.

Cells divide and migrate within an ECM that is rich in HA. Significant modulations of HA concentration and organization accompany cellular changes that take place during tissue and organ differentiation. Examples of dynamic events during which cells are surrounded by HA-rich ECM include: i) invasion of primary corneal stroma by mesenchymal cells to form mature cornea; ii) migration of cushion cells from the endocardium towards the myocardium during the formation of heart valves; iii) movement and proliferation of neuronal and glial cells during brain development; iv) division and migration of mesenchymal cells during embryonic limb development and fetal wound repair; and v) tumor cell growth and invasion. Several reported observations evidence the significant role of HA in cell division and migration.

During morphogenesis, the concentration and arrangement of pericellular HA can mediate cell-to-cell interactions that are essential for subsequent differentiation. As a result, HA-mediated cellular proliferation and migration leads to assembly of cells in appropriate numbers and positions during proliferation and differentiation of precursor cells into more mature tissues and organs. Interactions between HA and cell surface HA-binding proteins (HABPs) appear to have a distinctive role in binding of cells with HA, assembly of pericellular matrix, migration of endothelial cells, tubule formations, and angiogenesis (see e.g., Banerjee et al., 1991, Dev. Biol. 146:186-197; Banerjee et al., 1992, J. Cell Biol. 119:643-652).

For example, during angiogenesis, the tips of advancing capillaries are enriched in HA; such enrichment at capillary tips is not observed in non-growing capillaries. HA binding sites have also been detected on the surface of vascular endothelial cells. Liver sinusoids, which are known to be involved in clearance of HA from circulation, also exhibit HA binding sites.

An important way in which HA-rich matrix can promote cell proliferation is by provision of a hydrated pericellular zone that facilitates cell rounding during mitosis. HA synthase (HAS) is an enzyme that catalyzes synthesis of HA. HAS activity has been shown to fluctuate in coordination with the cell cycle and to peak during mitosis. Extrusion of HA onto the cell surface during mitosis can create a hydrated microenvironment that promotes partial detachment and rounding of the dividing cells. Inhibition of HA synthesis was shown to lead to cell cycle arrest at mitosis, just before cell rounding and detachment. HA-dependent pericellular matrix formation increases around dividing cells immediately before mitosis, and removal of this matrix by competitive displacement with HA oligosaccharides inhibits cell division.

The HA content of ECM can also influence the ability of a cell to migrate therethrough. A HA-containing matrix assembles around migrating cells, especially at the leading and trailing edges of the cells. For example, HA promotes glioblastoma cell migration within a fibrin gel by increasing hydration and, consequently, the porosity of the gel.

With respect to cell migration and invasion, HA-enriched matrices create hydrated pathways in cellular or fibrous barriers, thereby rendering those barriers susceptible to penetration by invading cells. Modulation of the density of these matrices by variation in the degree of binding between PGs and pericellular HA has been suggested to regulate cessation of cell migration (Knudson et al., 1993, FASEB J. 7:1233-1241). Several investigators have demonstrated that cell movement can be inhibited as a result of degradation of HA or by blocking the binding of HA with cell surface HA receptors CD44 or RHAMM (Ellis et al., 1997, Development 124:1593-1600; Thomas et al., 1992, J. Cell Biol. 118:971-977; Turley et al., 1993, Exp. Cell Res. 207:277-282; Boudreau et al., 1991, Dev. Biol. 143:235-247; Koocekpour et al., 1995, Int. J. Cancer 63:450-454). These observations indicate that, in addition to providing a suitable hydrated extracellular environment, interaction of extracellular HA with cell surface receptors can stimulate signaling pathways that promote cell movement or proliferation.

HA has a significant role in dynamic structural changes that occur within extracellular matrices during development and tissue remodeling, as well as its role in maintenance of mechanical properties and homeostasis of many tissues (see, e.g., Toole, 2001, Semin. Cell Dev. Biol. 12:79-87). Increased synthesis of HA is associated with wound repair, tumor invasion, and immune recognition. HA has been proposed to regulate cell locomotion and cytological differentiation that occur during these phenomena. HA can mediate these effects by sustained attachment of HA to HAS across the plasma membrane or by interacting with one or more receptors that specifically bind with HA (i.e., HA-binding proteins or HABPs).

In addition to extracellular HA, accumulating evidence indicates that HA is present in the cytoplasm and nuclei of cells in a number of tissues in vivo. Furthermore, changes in the distribution of intracellular HA and HABPs have been associated with proliferation of smooth muscle cells and fibroblasts (Evanko et al., 1999, Arterioscler. Thromb. Vasc. Biol. 19:1004-1013; Evanko et al., 1999, J. Histochem. Cytochem. 47:1331-1342).

Hyaluronic Acid-Binding Proteins (HABPs)

HABPs are sometimes referred to as “hyaladherins.” Several cell surface HABPs are known, including proteins designated CD44 (a trans-membrane receptor), TSG-6, and RHAMM (Receptor for HA-Mediated Motility) (Aruffo et al., 1990, Cell 61:1303-1313; Lee et al., 1992, J. Cell Biol. 116:545-557; Hardwick et al., 1992, J. Cell Biol. 117:1343-1350). Extracellular HABPs are also known, including the aggregating proteoglycans designated aggrecan, versican, brevican and neurocan (these are sometime referred to as the hyalectan or lectin family of proteoglycans; Iozzo, 1998, Annu Rev. Biochem. 67:609-652).

Most well-characterized HABPs have structurally similar HA-binding domains. These similar domains generally have amino acid sequence homologies in the range from 30-40% amino acid sequence identity. These domains are designated “LINK” modules or “proteoglycan tandem repeats” and form disulfide-bonded loops. LINK modules are known in the art and have been described in detail by others.

In many HABPs, two LINK modules are arranged in a tandem array. Two LINK modules form the HA-binding region of link proteins and the aggregating proteoglycans, whereas only a single LINK module occurs in the HA-binding domains of CD44 and TSG-6.

Some HABPs, such as RHAMM, do not have LINK modules. However, mutation and sequence-swapping studies with RHAMM showed a possible HA-binding motif that is present not only in RHAMM, but also within or adjacent to the link modules of other HABPs. The motif is B(X₇)B, where each B is an arginine or lysine residue and each X is, independently, any non-acidic amino acid residue. Variations of this motif (e.g., B(X₈)B) also bind HA with significant affinity. Clustering of basic amino acids within and around the motif appears to be the key aspect that determines binding. Examples of HABPs which contain the B(X₇)B and related sequence are ICAM, HA synthases, mammalian hyaluronidases, Cdc37 (a HA-binding cell cycle regulatory protein), P32 (a HABP that associates with splicing factors), and IHABP4 (an intracellular HABP).

Several proteins have been identified as intracellular HABPs, including CDC37, RHAMM/IHABP, and P32 (Entwistle et al., 1996, J. Cell. Biochem. 61:569-577; Assmann et al., 1998, J. Cell Sci. 111:1685-1694; Deb et al., 1996, J. Biol. Chem. 271:2206-2212; Huang et al., 2000, J. Biol. Chem. 275:29829-29839). Others have speculated that intracellular HA may have a role in regulating cellular behavior through interactions between intracellular HA and intracellular HABPs.

During mitosis, both HA and HABP molecules are present throughout the cytoplasm. HA and HABP molecules surround the chromosomes during their arrangement at the metaphase plate and during separation of the chromosomes in anaphase. This pattern is highly similar to the distribution of laminins during mitosis. HA is known to associate with chromatin, suggesting that HA has a role in chromosome condensation in at least some cells. Others have shown that rapid uptake of labeled HA and its subsequent accumulation in the cell, perinuclear area, and nucleus of transformed cells were associated with enhanced cell motility (Collis et al., 1998, FEBS Lett. 440:444-449).

HA is believed to influence cellular behavior through its interactions with HABPs. Consistent with this, many of the observed effects of HA on cell motility can be blocked by the antibodies which bind specifically with one of HABPs CD44, RHAMM, Cdc37, and p68 (Entwistle et al., 1996, J. Cell. Biochem. 61:569-577; Delpech et al., 1997, J. Intern. Med. 242:41-48; Toole 1997, J. Intern. Med. 242:35-40; Trochon et al., 1996, Int. J. Cancer 66:664-668).

Cancer and the Roles of Extracellular Matrix in Tumorigenesis

Neoplasia literally means “new growth.” It represents a pathological disturbance of cell growth characterized by excessive and unceasing proliferation of cells. Some neoplasms are designated benign because they grow slowly and remain localized so that the patient usually experiences little difficulty from them. Others are designated malignant, as they tend to proliferate rapidly and spread throughout the body, causing significant morbidity, mortality, or both.

In general, benign or non-cancerous tumors are slow-growing, innocuous and are usually of little pathological consequence to the host unless they secrete bioactive molecules, hemorrhage, or are physically located in such a way as to compromise the function of an organ (e.g., compressing the outlet of a gland). In contrast, malignant tumors have more rapid growth rates, often invading and eventually destroying adjacent tissues, and metastasize to distant sites. Malignant neoplasms range from well-differentiated to un-differentiated. Well-differentiated neoplasms resemble the normal cells from which they are derived. By contrast, undifferentiated or poorly-differentiated neoplasms are composed of undifferentiated (anaplastic) cells and/or cells which bear little or no resemblance to the normal cells from which they are derived.

Decreases in cellular differentiation are marked by a number of morphologic and functional changes. Anaplastic tumors characteristically display pleomorphism. Some cells may be many times larger while others can be extremely small and have a primitive appearance. The nuclei can be dark staining (hyperchromatic) and show increased nucleus:cytoplasm ratios, approaching 1:1 instead of the normal 1:4 to 1:6. Their chromatin is often coarsely clumped and distributed along with the nuclear membrane. Some anaplastic cells display unusual mitotic figures, sometimes producing multipolar (tripolar or quadripolar) mitotic spindles. The nucleoli are usually prominent. Another extraordinary feature of anaplasia is the formation of tumor giant cells, some possessing only a single huge nucleus, whereas others have two or more nuclei. In addition to these cytological abnormalities, the orientation of anaplastic cells is often markedly disturbed. Sheets or large masses of tumor cells grow in an anarchic, disorganized fashion. Although these growing cells obviously require blood supply, the connective tissue and vascular stroma are often scant and, in many anaplastic tumors, large areas experience ischemic necrosis.

Well-differentiated cancers resemble their tissue of origin. By way of examples, some well-differentiated adenocarcinomas of the thyroid gland form normal-appearing follicles, and some squamous cell carcinomas contain cells that do not differ cytologically from normal squamous epithelial cells. Thus, the morphological diagnosis of malignancy in well-differentiated tumors can sometimes be quite difficult.

The Roles of HA in Tumorigenesis and Cancer

HA is involved in regulation of several physiological and pathophysiological conditions, including cancer and carcinogenesis. Interactions between HA and HABPs appear to be important, or even necessary, in the tumorigenic and metastatic processes. Tumor cells migrate on, within, and through HA-containing matrices by interacting with the matrix by way of cell surface receptors, many of which are HABPs. The widespread occurrence of HA in substantially all human tissues suggests that the recognition of HA by one or more HABPs has an important role in tissue organization. Furthermore, overexpression of HA in many tumors of a wide variety of types further suggests a role for HA-HABP interactions in tumor states.

Interactions between HA and HABPs are known to be associated with malignancy. There are many HABPs that have been reported to have a role in carcinogenesis. In pathological states in adult tissues, such as in tumorigenesis, several characteristics behaviors are exhibited by endothelial cells, including those manifested during angiogenesis. These behaviors include lumen formation in ECM and cellular penetration therethrough, cellular migration, proliferation, and cell adhesion.

Previous investigators have demonstrated that many malignant solid tumors contain elevated levels of HA (see, e.g., Knudson et al., 1989, Ciba Found. Symp. 143:150-159; Knudson, 1996, Am. J. Pathol. 148:1721-1726). By way of example, HA levels have been shown to be elevated in lung tumors, Wilms' tumor and breast carcinomas (Knudson et al., 1989, Ciba Found. Symp. 143:150-159). High levels of HA expression correlate with poor differentiation in ductal carcinomas of human breast (Auvinen et al., 1997, Int. J. Cancer. 74:477-481) and with poor survival rates in human colorectal adenocarcinomas (Ropponen et al., 1998, Cancer Res. 58:342-347). It has been shown that urinary HA levels are elevated 2.5- to 6.5-fold in bladder cancer patients and that elevated urinary HA can serve as a highly sensitive and specific marker for detecting bladder cancer, regardless of tumor grade (Lokeshwar et al., 1997, Cancer Res. 57:773-777; Correction: 1998, Cancer Res. 58:3191, Lokeshwar et al., 2000, J. Urol. 163:348-356).

HA is believed to form a ‘halo’ around tumor cells that protects them against immune surveillance (Hobarth et al., 1992, Eur. Urol. 21:206-210). Enrichment of HA in tumors is believed to be attributable to increased production of HA by tumor cells themselves or to interactions between tumor cells and surrounding stroma, which induces increased production of HA by the stromal cells. Some malignant cells secrete or present membrane-bound agents that stimulate HA synthesis in adjacent fibroblasts (Knudson et al., 1984, Proc. Natl. Acad. Sci. USA 81:6767-6771).

It has been hypothesized by others that HA synthesis stimulated by cancer cells or stromal fibroblasts generates gaps extending through connective tissue, thereby creating space for movement of invading cancer cells (Knudson et al., 1989, Ciba Found. Symp. 143:150-159). Experimentally, it has been shown that elevated HA production by mouse mammary carcinoma cells and by melanoma cells correlates with elevated metastatic capacity (Kimata et al., 1983, Cancer Res. 43:1347-1354; Zhang et al., 1995, Cancer Res. 55:428-433). Up-regulation of HA production by stromal cells correlates with increased interaction between stromal cells and several types of malignant tumor cells (Knudson et al., 1989, Ciba Found. Symp. 143:150-159; Toole et al., 1979, Proc. Natl. Acad. Sci. USA 76:6299-6303; Knudson et al., 1984, Proc. Natl. Acad. Sci. USA 81:6767-6771; Asplund et al., 1993, Cancer Res. 53:388-392). Furthermore, HA accumulation has been demonstrated at the interface of tumor invasion into host tissues in various tumor types (Knudson et al., 1989, Ciba Found. Symp. 143:150-159; Wang et al., 1996, Am. J. Pathol. 148:1861-1869; Yeo et al., 1996, Am. J. Pathol. 148:1733-1740). Small fragments of HA (3-25 disaccharide units) are angiogenic and induce endothelial cell proliferation and migration with lumen formation (West et al., 1992, Exp. Cell Res. 183:179-196; Lokeshwar et al., 1997, Cancer Res. 57:773-777; Correction: 1998, Cancer Res. 58:3191; Banerjee et al., 1992, J. Cell Biol. 119:643-652). Each of these observations tends to support a correlation between HA synthesis and tumor cell malignancy.

Invasion and metastasis are biologic hallmarks of malignant tumors. In many cases, the mere existence of a tumor is not of great pathological significance, so long as the tumor does not physically or biochemically detriment other body organs or systems. In fact, small, stationary, benign tumors can persist for many years without ill effect. However, tumors which invade neighboring tissues or travel to distant body sites can cause significantly greater pathology. Such malignant tumors are the major cause of cancer-related morbidity and mortality.

Pathologically, metastasis is a pattern of tumor cell behavior that results from failure of some of the most fundamental regulatory processes controlling body organization. The metastatic process can be divided into two phases. The first phase is invasion of extracellular matrix by tumor cells and vascular (or other) dissemination of those cells within the body. The second phase is settling and establishment of tumor cells at body locations distinct from the tumor from which the cells derived. These metastatic processes occur naturally during development (e.g., during tissue and organ development in embryos) but, for most cell types, do not normally occur thereafter except under pathological conditions such as tumor metastasis.

Tissues are organized into a series of compartments separated from each other by two types of ECM components: basement membranes and interstitial connective tissues. Each of these ECM components is made up of collagens, glycoproteins, and proteoglycans. HA is another important constituent of these ECM components. Tumor cells must interact with the ECM at several stages in the metastatic process. Disruption of interactions between tumor cells and HA can disrupt the metastatic process at each of the stages at which such interactions occur.

Receptor-mediated attachment of tumor cells to laminin and fibronectin is important for invasion into ECM and metastasis. For example, over-expression of the basement membrane receptor laminin has been correlated by others with invasiveness in carcinomas of breast and colon. In addition to laminin-specific receptors, tumor cells also express integrins that can serve as receptors for many ECM components, including fibronectin, laminin, collagen, and vitronectin. Still others have observed a correlation between expression of alpha-4-beta-1 integrin (also known as VLA-4) on melanoma cells and their ability to metastasize. Receptor-mediated attachment of tumor cells to HA also appears to be important for tumor cell invasion into ECM and metastasis. HABPs have been implicated in tumorigenesis and metastasis.

The HABP CD44 is a widely distributed cell-surface glycoprotein that is encoded by a single gene but can be expressed as numerous isoforms as a result of alternative splicing. Published reports suggest that CD44-mediated events can enhance or inhibit tumor progression in different types of tumors. (Sy et al., 1991, J. Exp. Med. 174:859-866; Günthert et al., 1991, Cell 65:13-24; Iida et al., 1997, J. Cell Physiol. 171:152-160; Takahashi et al., 1995, Oncogene 11:2223-2232; Schmits et al., 1997, Blood 90:2217-2233). Others have reported that CD44 protein, particularly the 80-90 kDa isoform, has a role in metastasis of human tumor cell lines implanted in nude mice; a different isoform was required for metastatic behavior by rat pancreatic adenocarcinoma cells (Günthert et al., 1991, Cell 65:13-24). Analysis of CD44 mRNA extracted from urine pellets of human patients demonstrated that numerous abnormal CD44 transcripts occurred in 40 of 44 patients with bladder cancer, but not in people with neither urinary symptoms nor evidence of neoplasia (Matsumura et al., 1994, BMJ (Clinical Res. Ed.) 308:619-624). Overexpression of splice variants of CD44, containing epitopes encoded by exon 11 (v6) was correlated with poor prognosis in patients having colorectal carcinomas, breast carcinomas, non-Hodgkin's lymphomas, and other neoplasms (Mulder et al., 1994, Lancet 344:1470-1472; Kaufmann et al., 1995, Lancet 345:615-619; Koopman et al., 1993, J. Exp. Med. 177:897-904; Tarin, 1995, “Cancer metastasis,” In: Peckham et al., Eds., Oxford Textbook of Oncology, Oxford University Press, Oxford, pp. 118-132). These observations exemplify the role of HABP CD44 in tumor metastasis.

Expression of CD44H (the standard CD44 iso form) in CD44-negative human lymphoma cells or melanoma cells was shown to augment tumorigenicity in nude mice, and this effect was reportedly directly related to the ability of CD44 to bind HA (Sy et al., 1991, J. Exp. Med. 174:859-866; Bartolazzi et al., 1994, J. Exp. Med. 180:53-66). Similarly, metastasis by lymphoma cells to lymph nodes was reported to depend on interaction between CD44 and HA (Zahalka et al., 1995, J. Immunol. 154:5345-5355). Each of these effects attributed to CD44-HA interaction is mediated by intracellular signaling pathways. For example, in ovarian carcinoma cells, CD44 binds to the erbB-2/neu oncogene product and stimulates its kinase activity as well as cell growth (Bourguignon et al., 1997, J. Biol. Chem. 272:27913-27918). These observations further exemplify the role of HABP CD44 in tumorigenesis and tumor metastasis and suggest that other HABPs may have similar functions.

HA-RHAMM interactions have also been implicated in tumor cell behavior, both in vitro and in vivo. RHAMM is involved in the (intracellular) Ras and ERK signaling pathways and associates with the cytoskeleton. HA-RHAMM interaction induces transient phosphorylation of p125FAK in concert with turnover of focal adhesions in ras-transformed cells, thus leading to initiation of locomotion (Hall et al., 1995, Cell 82:19-28). Suppression of this interaction inhibits cell locomotion and proliferation in vitro and leads to inhibition of tumor growth in vivo. By contrast, over-expression of RHAMM leads to enhanced tumor growth and metastasis (Turley et al., 1001, J. Biol. Chem. 277:4589-4592; Hall et al., 1995, Cell 82:19-28).

Over-expression of cell surface RHAMM causes fibroblasts to become tumorigenic. Treatment of fibroblasts with a soluble form of RHAMM causes arrest of the cell cycle in G2/M, due to inhibition of Cdc2 and cyclin B1 expression, and inhibits fibrosarcoma growth and metastasis in vivo (Mohapatra et al., 1996, J. Exp. Med. 183:1663-1668). These characteristics of the HABP designated RHAMM further demonstrate the significance of HABPs in tumor metastatic processes.

The foregoing observations establish that interactions between HA and HABPs of cells undergoing tumorigenesis, metastasis, or both, have pathological significance in a wide variety of cancers.

Tumor Markers

A variety of cellular proteins have been identified by others as markers for the existence of tumors of various types. Immunohistochemical detection of many antigens in tumor tissue sections with various antibodies has been reported for various tumor markers. The following are some of the commonly used markers.

CA 27.29 and CA 15.3 are used as a marker for breast cancer with a sensitivity of 62% and 57% respectively. However these markers lack the required sensitivity and specificity for routine detection of cancer (Barbara et al., 1996, Lab. Medicine Newsletter 4(9)).

CA 19.9 is a monoclonal antibody raised against human colonic carcinoma cell line and recognizes a carbohydrate antigen. It is used as a marker for most pancreatobiliary adenocarcinomas and transitional cell carcinomas (Loy et al., 1993, Amer. J. Clin. Pathol., 993:726-728). However the false positive rate in patients with non-neoplastic diseases of pancreas, liver and biliary duct is 15% to 30% (Encabo et al., 1986. Bull. Cancer (Paris) 73:256-259).

CA 125 is a glycoprotein found in mucinous epithelial ovarian tumors and is recognized by monoclonal antibody OC 125 (Koelma et al., 1987, Histopathology 11:187-294). This antigen is also detected in cervix, endometrium, gastrointestinal tract and breast adenocarcinomas. OC 125 antibody is not useful for general tumor screening because, although it may have use as an antigen in ovarian cancer, ovarian cancers tend to be present at low incidence in the population and the CA 125 antigen appears to be expressed at high levels in many benign tissues (Barbara et al., 1996, Lab. Medicine Newsletter 4(9)).

CEA is a cell surface glycoprotein that has been shown by others to be useful as a marker for colorectal, gastrointestinal tract, lung, and breast carcinomas (Bates SE, and Longo DL, 1987, Sem. Oncology 14: 102-138). However, CEA is not useful for screening due to large numbers of false positive and negative results; also, many tumors do not produce CEA. (Barbara et al., 1996, Lab. Medicine Newsletter 4(9)).

CD44 is a transmembrane glycoprotein. Immunohistochemical and in situ hybridization studies have shown that over-expression of CD44v transcript localized only in cancer cells in colon adenocarcinomas (Gorham et al., 1996, J. Clin. Pathol. 49:482-488). The expression of splice variants containing epitopes encoded by v7-v8 is associated with poorer prognosis in cervical cancer patients (Angelos et al., 2002, Amer. Soc. Clin. Oncol., Abst. No. 868).

HMB 44 is a monoclonal antibody that identifies onco fetal glyco-conjugates in immature melanomas (Kapur et al., 1992, J. Histochem. Cytochem. 40:207-212). It also occurs in neural crest-derived tumors and in angiomyolipomas of the kidney (Friedman et al., 1991 Arch. Pathol. Lab. Med. 115:826-830).

AE-1, AE-3, CAM5.2 and 34bE12 are monoclonal antibodies that recognize low molecular weight and acidic cytokeratins and keratin subtypes that may help to determine the histogenesis of certain poorly differentiated neoplasms. Hepatocellular carcinomas are positive for AE3, and liver adenocarcinomas are positive for AE1. The antibody 34bE12 is used in the differential diagnosis between well-differentiated prostate carcinomas and their benign counterparts. The anti-keratin antibodies may also detect micro-metastases in bone marrow or lymph nodes.

Anti-p53 antibodies are used to detect mutation of the p53 tumor suppressor gene representing the most common genetic alteration in human tumors and can be detected with immunohistochemical techniques. Anti-p53 antibodies are also used to identify various tumor types, including colon, bladder, lung, breast and other carcinomas such as astrocytomas, leukemia's, sarcomas and mesotheliomas. Opitz et al., 2008, Eur. J. Cardiothorac. Surg. 33:502-502; Karsinskas et al., 2010, Mod. Pathol. 15 Jan. 2010 issue; Tajima et al., 2010, Oncogene 20:1941-1951.

The brief discussion of the foregoing antibodies and tumor markers shows the variability in the tumor markers and the inability of any single tumor marker to identify the occurrence of multiple tumor types.

A common tumor antigen, which can be used to detect substantially any tumor cell derived from substantially any source tissue in the human body has long been sought, previously unsuccessfully. Such a common tumor antigen would be useful for evaluation of potentially metastatic cells and for therapeutic applications. Such a common tumor antigen would be expressed by a wide spectrum of tumors, including both carcinomas and sarcomas from various human tissues, preferably irrespective of their origin.

It has previously been unknown whether an interaction between HA and a single HABP could be identified as informative of initiation, progression, and metastasis for multiple (or, more significantly, for substantially all) types of tumors. The subject matter described herein relates to monoclonal antibodies that bind specifically with a HABP that appears to be such a common tumor antigen.

### BRIEF SUMMARY OF THE DISCLOSURE

The disclosure relates to a monoclonal antibody designated MAbH11B2C2 that specifically binds with a hyaluronic acid-binding protein expressed by malignant human cells. The disclosure also relates to immunospecific portions of this monoclonal antibody. The antibody or immunospecific portion can be detectably labeled or have a cytotoxic agent (e.g., a radionuclide, a cytotoxin, a protein or polypeptide, an enzyme, a virus, or a combination of these) conjugated therewith.

The antibody or immunospecific portion can be used to assess malignancy of a human cell. Such methods are performed by contacting the cell with the antibody or immunospecific portion and assessing whether the cell and the portion specifically bind. Specific binding between the cell and the portion indicates that the cell is a malignant cell. Preferably, binding between the cell and the antibody/portion is compared with binding between a non-malignant cell of the same tissue type, so that expression of H11B2C2 antigen by the cell of interest can be compared with expression of the same antigen by the non-malignant cell. Enhanced expression of H11B2C2 antigen (as assessed by specific reactivity with the monoclonal antibody disclosed herein) by malignant cells of all or substantially all tissue types is disclosed herein. That is, the observation holds for at least brain cells, tongue cells, buccal mucosal cells, parotid cells, laryngeal cells, stomach cells, colon cells, duodenal cells, rectal cells, kidney cells, bladder cells, gall bladder cells, pancreatic cells, thyroid cells, breast cells, prostate cells, testicular cells, ovarian cells, endometrial cells, cervical cells, bone cells, and lymphatic cells. This observation and method can be employed in binding assays of substantially any known format (e.g., an ELISA or dot-blot format), including in vitro assays and assays in which either the cell being analyzed, the antibody/portion, or both are adhered to a surface.

In one embodiment, the disclosure relates to a method of assessing malignancy of a human sample cell. In this method, the sample cell is contacted with an immunospecific portion of monoclonal antibody MAbH11B2C2. Following such contact the following a) and b) are compared: a) the degree of specific binding between the immunospecific portion and the sample cell and b) the degree of specific binding between the immunospecific portion and a human control cell. The control cell is a cell of the same type as the sample cell and the control cell is known not to be a malignant tumor cell. A significantly greater degree of specific binding between the immunospecific portion and the sample cell than between the immunospecific portion and the control cell indicates that the sample cell is a malignant cell. The sample and control cells can, for example, be obtained from a single patient, or even be present in a single tissue sample obtained from the patient.

In another embodiment, the disclosure relates to a method of removing a human malignant cell from a suspension of cells. The method includes contacting the suspension with an immunospecific portion of monoclonal antibody MAbH11B2C2, linking the immunospecific portion with a substrate, and separating the substrate from the suspension after the suspension has been contacted with the immunospecific portion. The malignant cell remains specifically bound to the substrate and is removed from the suspension.

The disclosure also relates to a method of binding an agent with a human malignant cell. This method includes linking the agent with an immunospecific portion of monoclonal antibody MAbH11B2C2 and thereafter contacting the portion with the cell. The portion having the agent linked thereto specifically binds with the cell. The agent can, for example, be a detectable label or a cytotoxic agent.

Another aspect of the disclosure relates to a method of detecting the presence of a human malignant cell in a sample. The sample can, for example, be a body fluid obtained from a patient, such as blood, lymph, saliva, urine, sputum, mucus, a ductal lavage of breast, peritoneal fluid, a peritoneal lavage fluid, a bronchial lavage fluid, a vaginal secretion, a cervical scraping, semen, a colonic lavage fluid, or cerebrospinal fluid. The method includes contacting the sample with an immunospecific portion of monoclonal antibody MAbH11B2C2 and thereafter assessing whether any cell has bound with the immunospecific portion. Binding between a cell and the immunospecific portion is indicative that a malignant cell is present in the sample.

The disclosure also relates to an isolated human protein having a molecular size, as assessed by SDS-PAGE, of approximately 57 kDa, wherein the protein binds with hyaluronan and wherein the protein is specifically bound by monoclonal antibody MAbH11B2C2. The disclosure further relates to an isolated human protein having a molecular size, as assessed by SDS-PAGE, of approximately 37 kDa, wherein the protein binds with hyaluronan and wherein the protein is specifically bound by monoclonal antibody MAbH11B2C2.

In yet another aspect, the disclosure relates to a method of inhibiting infiltration of a malignant human cell into a hyaluronan-containing matrix. In this method, the cell is contacted and bound with an excess of monoclonal antibody MAbH11B2C2, so that substantially all MAbH11B2C2-binding sites on the cell surface are occupied by MAbH11B2C2. The ability of the cell to infiltrate the matrix is thereby inhibited.

The disclosure relates to a method of killing malignant human cells. The method includes the step of contacting the cells with an immunospecific portion of monoclonal antibody MAbH11B2C2 having a cytotoxic agent linked thereto. The immunospecific portion binds with the malignant cells and the cytotoxic agent kills the malignant cells. Such an agent-linked immunospecific portion can be administered to an animal having a malignant tumor. In such embodiments, it can be preferable that the agent be linked with the immunospecific portion by way of a linkage that is cleaved following internalization of the immunospecific portion by the cells.

### BRIEF SUMMARY OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 consist of three images, FIGS. 1A, 1B, and 1C, each of which illustrates HABP expression in a human carcinoma cell line, as shown by staining cells using MAbH11B2C2. FIG. 1A (50× magnification) depicts stained, sub-confluent breast carcinoma cells of cell line MCF-7. Both single cells and cell aggregates exhibited strong staining on the cell surface. FIG. 1B (100× magnification), depicts stained colon cancer cells of cell line 320 DM. Strong staining was observed on the cell surface, and some cells exhibited nuclear staining FIG. 1C (50× magnification), depicts stained prostate cancer cell line PC 3. Staining exhibited at the cell surface (arrow) demonstrated H11B2C2 expression at the cell surface.

FIG. 2 consist of two images, FIGS. 2A and 2B, each of which illustrates H11B2C2 expression in papillary carcinoma cells in a human thyroid gland tissue section, as shown by staining using MAbH11B2C2. FIG. 2A (40× magnification) depicts a tissue section stained using MAbH11B2C2. The arrow indicates tumor cells over-expressing H11B2C2 at the basal cell surface and surrounding cells. FIG. 2B (40× magnification) depicts a tissue section obtained from the same patient sample which was pre-incubated with 250 micrograms of HA polymer prior to staining using MAbH11B2C2, and illustrates inhibition of staining attributable to binding between HA and H11B2C2.

FIG. 3 consist of FIGS. 3A and 3B, each of which illustrates H11B2C2 expression in mixed parotid tumors. FIG. 3A (40× magnification) depicts a tissue section, stained using MAbH11B2C2, that exhibits over-expression of H11B2C2. The arrow indicates tumor cells exhibiting strong H11B2C2 expression, and the star indicates a region of stroma that was significantly less stained. FIG. 3B (40× magnification) depicts a tissue section from the same patient sample, stained using labeled non-specific antibody. No staining was observed.

FIG. 4 (40× magnification) illustrates H11B2C2 expression in infiltrating duct carcinoma of breast. H11B2C2 staining (i.e., expression) by MAbH11B2C2 was relatively intense in migrating tumor cells (arrow) and relatively low or non-existent in the stroma (star).

FIG. 5 consists of FIGS. 5A and 5B, each of which illustrates H11B2C2 expression in adenocarcinoma of stomach. FIG. 5A (25× magnification) depicts a gastric mucosa tissue section, stained using MAbH11B2C2, that exhibits relatively low expression of H11B2C2 in a region (arrow) in which benign tumor cells are present. FIG. 5B (25× magnification) depicts a gastric mucosa tissue section, stained using MAbH11B2C2, that exhibits relatively high expression of H11B2C2 in a region (arrow) in which malignant tumor cells are present.

FIG. 6 consists of FIGS. 6A and 6B, each of which illustrates H11B2C2 expression in moderately differentiated adenocarcinoma of pancreas. FIG. 6A (40× magnification) depicts a pancreas tissue section, stained using MAbH11B2C2, that exhibits relatively low expression of H11B2C2 in a region (arrow) containing benign acinar cells. FIG. 6B (40× magnification) depicts a pancreas tissue section, stained using MAbH11B2C2, that exhibits relatively high expression of H11B2C2 in a region (arrow) in malignant acinar cells. In FIG. 6B, low or no expression of H11B2C2 was observed in stroma (star).

FIG. 7 (25× magnification) illustrates H11B2C2 expression in poorly differentiated adenocarcinoma of gall bladder. H11B2C2 staining (i.e., expression), as assessed using MAbH11B2C2, is relatively intense at the cell surface and nucleus of the tumor cells (arrow) and relatively low or non-existent in the stroma (star).

FIG. 8 consists of FIGS. 8A and 8B, each of which illustrates H11B2C2 expression in transitional cell carcinoma of urinary bladder. FIG. 8A (40× magnification) illustrates H11B2C2 expression in a tissue section, stained using labeled MAbH11B2C2. Expression was relatively high in tumor cells (arrow), both at the nucleus and cell surface, and relatively low or non-existent in the stroma (star). The tissue section illustrated in FIG. 8B (40× magnification) was obtained from the same patient sample as in FIG. 8A and was stained with biotinylated secondary antibody, but not with MAbH11B2C2 to demonstrate lack of non-specific staining.

FIG. 9 (40× magnification) illustrates H11B2C2 expression in adenocarcinoma of colon. Strong expression of H11B2C2, as assessed using MAbH11B2C2, was observed for tumor cells, especially on the cell surface (arrow) and at the nucleus.

FIG. 10 (40× magnification) illustrates H11B2C2 expression in adenocarcinoma of rectum. Strong expression of H11B2C2, as assessed using MAbH11B2C2, was observed for tumor cells, especially at the cell surface (arrows).

FIG. 11 (40× magnification) illustrates H11B2C2 expression in adenocarcinoma of cecum. Strong expression of H11B2C2, as assessed using MAbH11B2C2, was observed for groups of tumor cells (arrow).

FIG. 12 consists of FIGS. 12A and 12B, each of which illustrates H11B2C2 expression in adenocarcinoma of prostate, stained using MAbH11B2C2. FIG. 12A (40× magnification) illustrates H11B2C2 expression in a tissue section including benign tumor cells. Expression was relatively low in cells in a hyperplastic area (arrow), and relatively low or non-existent in the stroma (star). The tissue section illustrated in FIG. 12B (40× magnification) was obtained from the same patient sample as in FIG. 12A and exhibited relatively high H11B2C2 expression in nests of tumor cells (arrow) infiltrated into the stroma, and relatively low or no expression in the stroma (star).

FIG. 13 (40× magnification) illustrates H11B2C2 expression in serous cyst adenocarcinoma of ovary. Strong expression of H11B2C2, as assessed using labeled MAbH11B2C2, was observed at the cell surface and nucleus of tumor cells (arrow).

FIG. 14 consists of FIGS. 14A and 14B, each of which illustrates H11B2C2 expression in carcinoma of uterine cervix. FIG. 14A (40× magnification) illustrates H11B2C2 expression in a tissue section, stained using labeled MAbH11B2C2. Expression was relatively high in squamous epithelial cells (arrow) and relatively low or non-existent in stroma (star). The tissue section illustrated in FIG. 14B (40× magnification) was obtained from the same patient sample as that shown in FIG. 14A and was stained with biotinylated secondary antibody (as was that shown in FIG. 14A), but not with MAbH11B2C2 to demonstrate lack of non-specific staining.

FIG. 15 (25× magnification) illustrates H11B2C2 expression in medulloblastoma cells stained using MAbH11B2C2. Strong expression of H11B2C2 was observed at the carrot shaped tumor cells (arrow).

FIG. 16 consists of FIGS. 16A and 16B, each of which illustrates H11B2C2 expression in Wilms' tumor (nephroblastoma) cells stained using MAbH11B2C2. FIG. 16A (25× magnification) illustrates H11B2C2 expression by Wilms' tumor cells (arrow). FIG. 16B (40× magnification) is a kidney tissue section (obtained from the same kidney from which the sample shown in FIG. 16A was obtained) with normal-looking glomerular structure (i.e., lacking Wilms' tumor cells) stained with MAbH11B2C2. Very low or no expression of H11B2C2 was observed. The arrow points to an area of normal glomerular structure.

FIG. 17 (40× magnification) illustrates H11B2C2 expression in osteosarcoma cells stained using MAbH11B2C2. Strong expression of H11B2C2 was observed in tumor cells (arrow), and relatively low or no expression was observed in the stroma (star).

FIG. 18 (40× magnification) illustrates H11B2C2 expression in seminoma cells of testis tissue stained using MAbH11B2C2. Strong expression of H11B2C2 was observed in the tumor cells (arrow) at the nucleus and cell surface.

### DETAILED DESCRIPTION

The disclosure relates to reagents and methods for assessing occurrence, malignancy, and metastatic potential of cells in humans. The same reagents and similar methods can be used to target delivery of agents (e.g., cytotoxic agents) to the same cells. The disclosure further relates to a protein herein designated H11B2C2 that is a hyaluronan binding protein (HABP) that is detectable using these reagents and methods.

### DEFINITIONS

As used herein, each of the following terms has the meaning associated with it in this section.

“H11B2C2” is a human protein having a molecular size of approximately 57 kDa (as assessed by SDS-PAGE) which binds with hyaluronan (HA) and which is specifically bound by monoclonal antibody MAbH11B2C2.

“HybH11B2C2” is the hybridoma clone designated HybH11B2C2, which was deposited under the provisions of the Budapest Treaty at ______ on ______ (deposit No. ______).

“MAbH11B2C2” is the monoclonal antibody produced by the HybH11B2C2 hybridoma. Except where context clearly indicates otherwise, “MAbH11B2C2” refers alternatively to this monoclonal antibody or to an immunospecific portion thereof.

An “immunospecific portion” of an antibody is a fragment of the antibody that specifically binds with the antigen of the antibody and includes either or both of a VL and a VH domain of the antibody. Non-limiting examples of such fragments include Fab, Fab′, F(ab′)2, and Fv fragments, as well as single-chain antibodies, single-chain Fvs (scFv), and disulfide-linked Fvs (sdFv). Immunospecific portions of an antibody, including single-chain antibodies, may include the variable region(s) alone or in combination with the entirety or a portion of the following: hinge region, CL, CH1, CH2, and CH3 domains.

An antibody or fragment thereof “specifically” binds with a ligand if it binds the ligand with the specificity normally associated with antibody-antigen binding in the field of immunology—e.g., with a Kₐ of greater than or equal to about 10⁴ M⁻¹, preferably of greater than or equal to about 10⁵ M⁻¹, more preferably of greater than or equal to about 10⁶ M⁻¹, and still more preferably of greater than or equal to about 10⁷ M⁻¹. Affinities of binding partners can be readily determined using conventional techniques, for example those described by Scatchard et al., Ann. N.Y. Acad. Sci. 51:660 (1949).

The term “malignant” is used in its oncological sense, referring to non-benign tumor cells.

### DESCRIPTION

This disclosure relates to a human HA-binding protein that has been discovered to be an apparently universal marker of malignancy in human tumor cells. This protein, designated H11B2C2 exists in at least two forms, one having a molecular size of approximately 57 kilodaltons (kDa) and the other having a molecular size of approximately 37 kDa, (each as assessed by SDS-PAGE). Substantially enhanced expression, relative to non-tumor cells of the same type, of at least the 57 kDa form was detected in malignant tumor samples of all types tested—not fewer than thirteen different types of malignant human tumors. Furthermore, substantial expression of H11B2C2 was detected in at least three human cancer cell lines. These data indicate that H11B2C2 is expressed at relatively high levels in all, or substantially all, human malignancies.

Expression of H11B2C2 in a human breast tumors was reported earlier in a non-enabling disclosure (Kumarswamy et al., 2008 Adv. Biol. Res. 2:6-12) that did not disclose the identity of the protein or any repeatable method of isolating or identifying it. Also disclosed, but not enabled, was a monoclonal antibody herein designated MAbH11B2C2 that specifically binds with H11B2C2 and a hybridoma, herein designated HybH11B2C2, that produces that antibody.

MAbH11B2C2 is a reagent that binds specifically with H11B2C2 and, as such, can be used as a reagent for binding any cell that expresses H11B2C2. For cells that express this protein on their surface, MAbH11B2C2 can be bound directly to the surface of the cells. For cells that express H11B2C2 internally (e.g., on the nuclear surface), MAbH11B2C2 can be used to bind the protein in samples (e.g., tissue sections) in which the interior of the cell is made accessible to MAbH11B2C2. As is well known, fragments of antibodies can be prepared that include less than the entire amino acid sequence (and fewer than all polypeptide chains) of an antibody, and these fragments will retain some or all of the antigen-binding properties of the corresponding complete antibody so long as at least part of the complementarity-determining variable region(s) of the antibody are retained. Similarly with MAbH11B2C2, antibody fragments can be prepared using known methods, the fragments relevant to this disclosure being those that retain an immunospecific portion of MAbH11B2C2, such that the portion binds specifically with H11B2C2.

MAbH11B2C2, or an immunospecific portion thereof, can be used to identify cells that express H11B2C2 and, furthermore, to estimate or quantify the degree to which this protein is expressed by cells. It has been discovered that expression of H11B2C2, as assessable using MAbH11B2C2 or an immunospecific portion thereof, is an indication that a cell is a malignant cell. This disclosure includes methods of using MAbH11B2C2 to assess the malignant state of cells (or to assess that cells are tumor cells), to reversibly bind (i.e., adhere) malignant cells to a substrate, and to bind (i.e., deliver) a wide variety of agents to malignant cells. By way of example, a cytotoxic agent can be linked with this antibody in order to induce death in cells to which the antibody specifically binds, or cells to which the agent-linked antibody binds at high density (i.e., cells which express H11B2C2 at a relatively high level).

Antibodies and Fragments Thereof

The disclosure includes MAbH11B2C2 and immunospecific portions of this monoclonal antibody. Each of these molecules specifically binds with HA-binding protein H11B2C2, which is expressed by malignant human cells. The antibody or fragment can have one or more other entities bound, fused, or linked therewith, so long as the other entity does not substantially alter the H11B2C2-binding specificity of the antibody or fragments. By way of example, cytotoxic agents, detectable labels, and coupling moieties (e.g., biotin moieties) can be linked therewith.

The antibodies and fragments described herein can be used alone or in combination with other compositions. The antibodies and fragments can be recombinantly fused to a heterologous polypeptide at one or both of their amino- and carboxyl-termini, or they can be chemically conjugated (covalently or non-covalently) with polypeptides or other moieties. For example, antibodies of the present invention may be recombinantly fused or conjugated to molecules useful as labels in detection assays and effector molecules such as heterologous polypeptides, drugs, or toxins. See, e.g., PCT publications WO 92/08495; WO 91/14438; WO 89/12624; U.S. Pat. No. 5,314,995; and EP 396,387.

The antibodies and fragments described herein include derivatives that are modified, i.e., by the covalent attachment of any type of molecule to the antibody such that covalent attachment does not prevent the antibody from exhibiting an anti-idiotypic response. For example, the antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative may contain one or more non-classical amino acids.

MAbH11B2C2 can be produced by hybridoma HybH11B2C2 using well known methods. Antibody fragments that immunospecifically bind with H11B2C2 can be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., Proc. Natl. Acad. Sci. USA 90:7995-7999 (1993); and Skerra et al., Science 240:1038-1040 (1988).

Chimeric antibodies can be made by joining one or more antigen-binding variable domains of a mouse monoclonal antibody (e.g., one or more variable domains of MAbH11B2C2) to one or more constant domains of human antibodies. For example, the V_{L} domain of MAbH11B2C2 can be joined with a human C_{L} domain and the V_{H} domain of MAbH11B2C2 can be joined with human CH1, CH2, and CH3 antibody domains (e.g., with these domains linked sequentially to one another, i.e., CH1-CH2-CH3) to make the light and heavy chains, respectively, of a chimeric antibody. These methods are well known (e.g., Boulianne et al., 1984, Nature 312:643-646; Morrison et al., 1984, Proc. Natl. Acad. Sci. USA 81:6851-6855).

Humanized antibodies can be made using known methods by grafting the antigen-binding loops known as complementary determining regions (CDRs) of a mouse monoclonal antibody such as MAbH11B2C2 into a human IgG framework. (e.g., as described in Riechmann et al., 1988, Nature 332:323-327). Alternatively, the technique called-Phage display can be used to generate humanized antibodies. This technique involves ligation of a gene encoding a human antibody into the genome of a bacterial phage and allowing phages so generated to infect their host bacteria, so that the bacteria display the human antibody (see, e.g., Vaughan et al., 1998, Nature Biotechnol. 16:535-539). By immobilizing purified H11B2C2 protein obtained from human tumor tissue to the surface of a well (for example ELISA plate wells), a phage that displays an antibody that binds specifically with H11B2C2 will remain bound to the well, while others can be removed by washing. This phage display method can be used both with full-length H11B2C2 protein and with specific epitopes thereof (e.g., the epitope of H11B2C2 protein with which MAbH11B2C2 binds) to produce fully human antibodies. Still another method that can be used to produce fully human monoclonal antibodies that bind specifically with H11B2C2 protein involves use of transgenic mice. This method involves 1) inactivating endogenous mouse IgG genes, 2) introducing human IgG gene segments into the mouse germ line, and 3) immunizing the transgenic mouse with H11B2C2 protein isolated from human cancer tissue. (see, e.g., Lonberg, 2005, Nature Biotechnol. 23:1117-1125; Vaughan et al., 1998, Nature Biotechnol. 16:535-539).

In addition to detectable labels, MAbH11B2C2 and immunospecific portions thereof can have a wide variety of molecules attached thereto, as is well known in the field of immunology.

The identity of a detectable label used in connection with the antibody and antibody fragments described herein is immaterial, as a wide variety of detectable moieties are know to be useful for rendering antibodies and fragments detectable. A few examples of such detectable labels include directly-detectable markers, such as radionuclides and chromophores, and indirectly-detectable labels, such as fluorophores and enzymes. Other examples of useful labels include proteins that bind with a specific chemical entity (e.g., an avidin, such as streptavidin, which binds specifically with biotin, with either the biotin or the avidin being linked with the antibody). Yet other examples of useful labels include antibodies that bind with MAbH11B2C2, including antibodies which binds specifically with MAbH11B2C2 and antibodies that bind with all mouse-derived antibodies (MAbH11B2C2 is derived from a murine hybridoma). That is, substantially any known antibody-detection techniques or reagents can be used to detect MAbH11B2C2.

A wide variety of cytotoxic agents can be conjugated with MAbH11B2C2 using known reagents and technologies. Such cytotoxic agents can be conjugated with the antibody using cleavable linkage technologies, such that the agents are separated from the antibody upon delivery of the antibody conjugate to a cell. Preferably, such cleavable linkages are selected so that the agent is deconjugated from the antibody (e.g., by cleavage of an agent-antibody linkage by a lysosomal enzyme) following internalization of the conjugate by a cell. By way of example, small molecule cytotoxins such as maytansinoids, calicheamicin, and auristatins can be conjugated with this (or other) antibodies using known technologies. Substantially any known methods or reagents for conjugating cytotoxic molecules (including fusing cytotoxic proteins) with antibodies can be used to prepare conjugates (including fusion proteins) with MAbH11B2C2.

Uses of MAbH11B2C2

The monoclonal antibody MAbH11B2C2 and immunospecific portions thereof can be used to achieve a wide variety of purposes, including substantially any purpose involving binding or associating a moiety with H11B2C2 protein or with a cell, or a component of a cell, that expresses H11B2C2.

By way of example, MAbH11B2C2 and immunospecific portions thereof can be used simply as a reagent for detecting H11B2C2. Because of the association disclosed herein between expression of H11B2C2 and the malignant (and tumorous) status of cells, these reagents can be used as diagnostic reagents for assessing the presence of malignant (or tumorous) cells in a sample, such as a tissue sample (e.g., blood or biopsied solid tissue) taken from a patient. Likewise, MAbH11B2C2 can be used (e.g., using common affinity chromatography techniques) to isolate or purify H11B2C2 protein (or cells which express it on their surface) from a mixture, such as a cell lysate or a suspension of cells.

As described herein, MAbH11B2C2 and immunospecific portions thereof inhibit binding between H11B2C2 and HA. The antibodies and portions thereof can therefore be used to inhibit such binding in a variety of situations. By way of example, these reagents can be used to inhibit binding between cells that express H11B2C2 and HA or HA-containing extracellular matrix materials, either in vitro or in vivo. Similarly, HA and oligomers of HA (e.g., oligomers containing 3-25 disaccharide units) can be used as a reagent to compete with binding of MAbH11B2C2 with H11B2C2, for example to estimate the concentration of H11B2C2 in a suspension or on a cell surface using a competition assay.

MAbH11B2C2 and immunospecific portions thereof can be used to bind cells that express H11B2C2 to another entity. For instance, if the antibody or portion is attached to a substrate, cells expressing H11B2C2 can be bound to the substrate by permitting the portion of the substrate having the antibody attached thereto to contact the cells. Thus, for instance, cells that express H11B2C2 can be separated from a mixture of cells that includes cells that do not express H11B2C2 by contacting the mixture with the substrate and then separating the substrate from the mixture. If an excess of antibody is present (relative to the number of H11B2C2-expressing cells in the mixture) and the substrate and mixture are thoroughly contacted, complete or near-complete removal of H11B2C2-expressing cells from the mixture can be achieved.

In an important embodiment, MAbH11B2C2 or an immunospecific portion thereof has a cytotoxic agent attached thereto, such that when the agent is brought into close proximity with any cell to which the antibody/portion binds, it induces or contributes to death of the bound cell. Owing to the specificity of H11B2C2 expression by tumorous and malignant cells, such methods can be used to specifically target such cells for death, either in vitro or in vivo.

In another important embodiment, MAbH11B2C2 or an immunospecific portion thereof has a detectable label attached thereto, such that when the antibody binds with H11B2C2 (e.g., protein immobilized on a membrane or a cell or tissue that expresses the protein on its surface), the label becomes immunospecifically associated with the protein. Such methods are useful, for example, in detecting and spatially visualizing occurrence of H11B2C2.

MAbH11B2C2 or an immunospecific portion thereof can be used to target cancer stem cells. Cancer stem cells are believed to be important for initiation, maintenance and recurrence of various malignancies. The cancer stem cells have been identified in several tumors such as those of brain, breast, colon, ovary, pancreas, and prostate, and in leukemia. Others have suggested that leukemia stem cells could be targeted in their niche (‘niche’ referring to the in vitro or in vivo stem cell microenvironment) using antibodies specific for antigens (such as CD44 and CD123, each of which is a HABP) expressed on the surface of stem cells, which are necessary for homing and survival (Lane et al., 2009, Blood. 114(6):1150-1156). Similarly, MAbH11B2C2 can be used stop tumor growth by blocking the interaction between cancer stem cells and surrounding extracellular matrix.

Other uses of MAbH11B2C2 and immunospecific portions thereof are apparent to skilled artisans in the fields of immunology and oncology, for example.

Diagnostic Methods

MAbH11B2C2 and immunospecific portions thereof (herein “MAbH11B2C2” or “the antibody”) can, for example, be used to detect occurrence of H11B2C2 in a wide variety of samples. In such methods, the antibody is made detectable by linking a detectable label, or a moiety that can be associated with a detectable label, with the antibody. Upon binding of the antibody with H11B2C2 protein (i.e., under conditions suitable for antibody-antigen binding), the label or label-associable moiety becomes immunospecifically associated with the protein. The sample can be rinsed to remove label not immunospecifically associated with the protein, if desired. Detection of the protein-bound label in the sample indicates occurrence of H11B2C2 in the sample. Depending on the label-detection methods used, the location of the label within the sample (i.e., physical location in a microscopically-observed tissue section) indicates the location at which H11B2C2 occurs. Thus, occurrence of this protein can be geometrically determined within a sample.

MAbH11B2C2 can be used in a method of assessing malignancy of a human cell. In methods of this type, the cell is contacted with MAbH11B2C2 (or with an immunospecific portion thereof). Thereafter, and preferably after rinsing the cell under conditions sufficient to substantially remove any antibody not immunospecifically bound to the cell, one assesses whether immunospecific binding has occurred between the cell and the antibody. Occurrence of specific binding between the cell and the antibody is an indication that the cell is a malignant cell. This assay can be performed in substantially any of the nearly innumerable immunological assay formats that are known, such as an ELISA format or an immunoblot format involving detection of radio labeled antibody.

In this method, the identity of the cell is not critical. As described in the examples disclosed herein, the operability of this method has been demonstrated with all (13+) human malignant tumor samples that have been tested, as well as with several human tumor cell lines. Examples of suitable malignant cell types that can be assessed using this method include brain cells, tongue cells, buccal mucosal cells, parotid cells, laryngeal cells, stomach cells, colon cells, duodenal cells, rectal cells, kidney cells, bladder cells, gall bladder cells, pancreatic cells, thyroid cells, breast cells, prostate cells, testicular cells, ovarian cells, endometrial cells, cervical cells, bone cells, and lymphatic cells. The source of the cells is likewise not critical. Cells taken directly from a human patient can be assessed, as can cells from archived (e.g., frozen, paraffin-embedded, or lyophilized) samples.

This diagnostic method can be performed in vitro or in vivo, as the ability of the antibody to bind with a malignant cell should not depend on the physical location of the cell, so long as the antibody and the cell are in fluid communication with one another. In a convenient embodiment, a sample is obtained from an individual patient and occurrence of H11B2C2 in the sample is assessed as described herein. Occurrence of H11B2C2 in the sample is an indication that one or more malignant cells is present in the sample. The sample can be a fluid sample such as blood, lymph, saliva, urine, sputum, mucus, a ductal lavage of breast, peritoneal fluid, a peritoneal lavage fluid, a bronchial lavage fluid, a vaginal secretion, a suspended cervical scraping, semen, a colonic lavage fluid, and cerebrospinal fluid. Alternatively, the sample can be a solid tissue sample, such as a tumor biopsy, a cervical smear, or a resected tumor.

When performed in vitro, the diagnostic methods can be performed in a wide variety of formats, as are well known in the field of antibody-based diagnostic methods. By way of a first example, MAbH11B2C2 can be fixed to a substrate, the sample contacted with the substrate, the substrate rinsed to remove non-immunospecifically-binding materials, and cells or H11B2C2 bound to the substrate can be detected. By way of a second example, components of a sample can be fixed to a substrate, MAbH11B2C2 contacted with the substrate, the substrate rinsed to remove non-immunospecifically-binding materials, and MAbH11B2C2 bound to the substrate can be detected. By way of a third example, MAbH11B2C2 and the sample can be contacted by combining them in a common fluid, and formation of immunospecifically-bound complexes of MAbH11B2C2 and components of the sample can be detected, for example by binding such complexes with a substrate or by assessing agglutination in the fluid. Selection of an appropriate format is within the ken of a skilled artisan in this field, taking into account such factors as the identity and nature of the sample to be tested, the characteristics of malignant cells expected to be observed in the sample, and the desired speed and throughput of the diagnostic assay.

The diagnostic assays described herein can be performed alone or in conjunction with other diagnostic assays for assessing carcinogenic, malignant, or metastatic state. By way of example, a sample obtained from a patient can be divided into multiple portions, with one portion assayed according to the diagnostic methods described herein and other portions assayed according to other diagnostic methods (e.g., assessment of CA125 or p53 expression). The diagnostic assays described herein can be performed in conjunction with traditional histological analyses of cells and tissue samples. By way of example, cells obtained in an ordinary Pap smear procedure are fixed on a glass slide, and observed microscopically after staining Prior to, after, or simultaneously with such staining, the cells can be contacted with (e.g., fluorescently) labeled MAbH11B2C2. Visual light microscopic examination of the stained sample can be performed per normal Pap smear procedure to identify cells exhibiting abnormal cytology. In addition, fluorescent microscopic examination of the same sample can be performed to identify cells that express H11B2C2 on their surface. The results can be considered separately or, preferably, in conjunction, with cells which both exhibit both abnormal cytology and H11B2C2 expression being particularly indicated as likely malignant cells.

In an important embodiment, the diagnostic methods described herein are used to detect a particular malignancy, such as prostate cancer. In these methods, cells of a selected type (e.g., prostate cells obtained by biopsy) are obtained from a human patient and reactivity with MAbH11B2C2 of those cells is compared with reactivity with MAbH11B2C2 of cells of the same type obtained from a “control” individual known not to be afflicted with the particular malignancy, or from a population of such individuals. If the reactivity of the cells obtained from the patient is significantly greater that the reactivity of the cells obtained from the control individual (or population), then this is an indication that the patient's cells included malignant cells and that the patient is afflicted with the particular malignancy. In place of cells from the control individual, cells from a non-malignant cell line or data characterizing H11B2C2 expression in non-malignant cells of the selected type can be used. A skilled artisan recognizes that selection of the cell type to be obtained from the patient and assessed corresponds to the particular malignancy to be detected.

In another important embodiment, a patient sample (e.g., blood or peritoneal fluid) which can harbor cells from multiple potential tissues-of-origin is assessed using the diagnostic methods disclosed herein. Detection of a greater degree of expression of H11B2C2 than is detected in a corresponding sample from a control individual not afflicted with a malignancy of any the potential tissues-of-origin is an indication that the patient is afflicted with a malignancy of one or more of those tissues. Even though this method may not identify a single tissue as the origin of the malignancy, it is informative in that it indicates that at least one malignancy is present among the potential tissues-of-origin.

Because malignant cells are necessarily tumor cells, the diagnostic methods described herein can be used to assess whether human tumor cells occur in a sample. In these methods, the sample (or a portion thereof) is contacted with an immunospecific portion of monoclonal antibody MAbH11B2C2. Binding of the antibody with a cell in the sample indicates that the sample includes human tumor cells.

Furthermore, the extent to which MAbH11B2C2 binds with a cell can be used to differentiate malignant tumor cells from other cells. Binding of MAbH11B2C2 to a cell surface is proportional to the level of H11B2C2 protein expression at the cell surface, and it has be discovered that the degree of MAbH11B2C2 to malignant tumor cells is substantially not tissue- or cell-type-specific. That is, the monoclonal antibody can be used to assess malignancy in any of a wide variety of cell types. Relative levels of MAbH11B2C2 binding can be assessed using any of a variety of known techniques for assessing antibody binding to cell surfaces. For example, commercially available software such as Adobe PHOTOSHOP™ brand image manipulation software can be used in conjunction with software written for assessment of staining in histopathology slides, such as that described in J. Histochem. Cytochem 2000; 48:303-311. World Wide Web-based image analysis systems, such as the ImageJ software available through the U.S. National Institutes of Health can be used for image analysis, as can commercial image analysis software packages, such as the Image Lab™ brand software package available from Bio-Rad Corporation. We have consistently observed that malignant tumor cells express higher H11B2C2 expression levels (and, consequently, higher MAbH11B2C2 staining levels) than benign tumor cells or normal (i.e., non-tumor) cells. For at least some cell types, differences in H11B2C2 protein expression (as assessed by MAbH11B2C2 staining) between benign tumor cells and non-tumor cells could not be detected.

Thus, significant binding of a cell by MAbH11B2C2 indicates that the cell is malignant.

The diagnostic methods described herein can be used to assess the tumor status of a patient or sample whose tumor status was not previously known. The methods may also be used to monitor the tumor status of a patient whose tumor status was known at an earlier time. By way of example, the tumor status of a patient who was known to be afflicted with a malignant tumor can be assessed after a course of therapy to determine whether malignant cells remain detectable in the patient and, if so, at what level. The methods may thus be used to assess the efficacy or progression of an anti-cancer therapy.

Inhibiting Interactions Between Malignant Cells and Hyaluronan

As described in the examples herein, monoclonal antibody MAbH11B2C2 inhibits binding between protein H11B2C2 and hyaluronans. Because malignant cells express H11B2C2 at higher levels than non-malignant cells of the same type, MAbH11B2C2 can be expected to interfere with interactions between malignant cells and HA-containing extracellular matrices (ECMs) to a greater degree than it interferes with interactions between normal cells and ECMs. Furthermore, because interactions between malignant cells and ECMs (including HA-containing ECMs) is believed to have a significant role in growth of tumors, invasion of neighboring tissues by tumors, and metastasis of tumor cells, MAbH11B2C2 can be used to inhibit these tumor processes.

MAbH11B2C2 can be used to inhibit infiltration of a malignant human cell into a HA-containing matrix. In such methods, the cell is contacted with an immunogenic portion of monoclonal antibody MAbH11B2C2, which occupies H11B2C2 moieties that could otherwise interact with HA in the matrix. The ability of the malignant cell to infiltrate into the matrix is thereby inhibited. As greater quantities of MAbH11B2C2 are used, a greater fraction of cellular H11B2C2 moieties will be occupied with MAbH11B2C2, a smaller fraction of those moieties will be available for interaction with HA in the matrix, and the ability of the cell to infiltrate into or through the matrix will be inhibited to an increasing degree. In one embodiment, an excess (relative to the number of H11B2C2 moieties on the cell surface) of MAbH11B2C2 is contacted with the cell, such that substantially all H11B2C2 moieties on the cell surface are occupied by MAbH11B2C2 and the ability of cell-surface H11B2C2 to contribute to interaction with the matrix by the cell is substantially eliminated. For malignant cells having the ability to infiltrate an HA-containing matrix substantially only by virtue of cell-surface H11B2C2, such treatment would effectively substantially eliminate the ability of the cell to migrate or grow into (or through) such matrices.

MAbH11B2C2 therefore has use as an agent for inhibiting, reducing, slowing, or eliminating the ability of a malignant cell to migrate into or through an HA-containing matrix. Many tissues susceptible of malignancy are separated from other tissues and body cavities (such as the lumens of nearby blood vessels) by HA-containing matrices. MAbH11B2C2 can be contacted with a malignant human cell in vivo, and such contact will result in inhibition of the ability of cell-surface H11B2C2 to interact with HA in matrices that separate that malignant cell from other cells, tissues, and body cavities. By interfering with these interactions, MAbH11B2C2 can be used to inhibit infiltration of malignant human cells into ECM that surrounds the malignant cells, thereby inhibiting, reducing, slowing, or eliminating the ability of the malignant cells to migrate from one location in a human's body to another location. Inhibiting, reducing, slowing, or eliminating malignant cell movement with a human's body can inhibit growth of tumors, invasion of tumors into surrounding tissues, and metastasis of human tumor cells.

The ability of each of MAbH11B2C2 and HA to inhibit binding of the other with H11B2C2 renders each of these a suitable reagent for measuring concentrations (and surface densities) of the two components using substantially any known competitive inhibition-based quantification assay. In one type of such assays, binding between the target (here, H11B2C2) and one of the reagents (here, MAbH11B2C2 and HA) is assessed in the presence of one or more known concentrations of the other reagent, and the amount of binding of the one or both of the reagents to the target are assessed, whereby the concentration of the unknown reagent can be deduced from binding of the known reagent. In an alternative type of such assay, the reagents are contacted with the target (e.g., a known amount of the target), and the amount of binding of the one or both of the reagents to the target are assessed.

Therapeutic Methods

MAbH11B2C2 (or an immunospecific portion thereof) can be used as a treatment for humans afflicted with malignant tumors, either alone or in combination with other tumoristatic or tumor-reducing therapies. Such therapeutic methods can take several forms.

In one form, MAbH11B2C2 is used simply as a tumoristatic agent, binding H11B2C2 moieties on the surface of tumor cells, preventing those tumor cells from interacting with HA in ECM, and thereby preventing the tumor cells from growing into or infiltrating the ECM. In such methods, the antibody is contacted with the tumor cells, for example by injecting a suspension of the antibody into a tumor mass or into a body fluid (e.g., blood or peritoneal fluid) that contacts tumor cells. Binding of the antibody with H11B2C2 on tumor cells blocks the ability of H11B2C2 to bind with HA, thereby reducing the ability of the tumor cells to interact with HA. Reducing the ability of tumor cells to interact with HA can reduce the ability of tumor cells to grow, replicate, or migrate into or through ECM.

In another form of tumor-reducing therapy, MAbH11B2C2 is used as a reagent to reduce tumor metastasis before, during, or after biopsy, resection, or other surgical manipulation of a tumor. The antibody is contacted with tumor cells, for example by injecting a suspension of the antibody into a tumor mass or into a body fluid that contacts tumor cells. The antibody binds with H11B2C2 on tumor cells, which blocks the ability of H11B2C2 to bind with HA. In this way, one can reduce the ability of tumor cells dislodged from a tumor mass during surgical manipulation to bind to or invade body sites adjacent to, or distant from, the original tumor site. Because the antibody can bind only H11B2C2 on surfaces to which the antibody has fluid communication, it is advisable that the antibody be contacted with newly-exposed surfaces (e.g., at the site of a surgical incision). This can be achieved by exposing the surface in the presence of a suspension of the antibody or by rinsing the newly-exposed surface with such a suspension prior to making any further surgical manipulation, for example.

Anti-metastatic doses of MAbH11B2C2 can be administered with a physiologically acceptable carrier such as saline solution to the affected tumor area (or, perhaps less preferably, systemically) so that the antibody can specifically inhibit tumor cell migration. The doses given in specific cases will depend on the method of application, the patient's tumor burden, the other needs of the patient, and other factors within the level of skill in the field of tumor therapy. Doses, timing, and other therapeutic characteristics can be adjusted based on the patient's response.

Because MAbH11B2C2 is a murine antibody (i.e., derived from a murine hybridoma), it can be expected that the immune system of a human patient to whom the antibody (or an immunospecific portion thereof) is administered will mount an immune response to eliminate the murine antibody and any cells to which it is bound. As described herein, MAbH11B2C2 binds immunospecifically with tumor cells, and particularly with malignant tumor cells. Thus, administration of MAbH11B2C2 to a patient who harbors tumor cells can induce an immune response in the patient that will attack the tumor cells. MAbH11B2C2 can be administered to a human patient for the purpose of inducing such an immune response, for example to shrink or eliminate a tumor. In such a method, the antibody is administered to a tumor tissue or to a body fluid or cavity that is in fluid communication with tumor cells.

If a humanized form of the antibody is administered to such a patient, then the anti-murine-antibody response of the immune system can be dampened or eliminated and binding of the humanized antibodies with tumor cells can induce a humoral immune response (e.g., activation of the complement system) in the vicinity of antibody-bound tumor cells. Inflammation and other immune-mediated phenomena that are induced in the vicinity of antibody-bound tumor cells can induce attack upon, and death of, neighboring cells, including tumor cells that may not have the antibody bound thereto. In this way, administration of the antibody can induce immune attack upon not only the tumor cells to which it binds, but also upon neighboring tumor cells.

Rather than relying upon recognition by a patient's immune system of MAbH11B2C2 as a foreign immune target (or as an antigen-bound ‘self’ antibody triggering humoral immunity), MAbH11B2C2 (or an immunospecific portion thereof) can be used as a carrier to deliver to tumor cells a cytotoxic agent. This can be achieved using an antibody having the cytotoxic agent conjugated therewith. This can also be achieved using an antibody having a moiety with which the cytotoxic agent can be selectively bound after the antibody has bound to a tumor cell. In either instance, association of the cytotoxic agent with a tumor cell induces death of the tumor cell. A wide variety of cytotoxic agents and technologies for associating them with an antibody are known, and substantially any of these can be used in the therapeutic methods.

In instances in which a solid tumor mass is known or believed to exist substantially well-delineated from surrounding non-tumor tissue, cytotoxic agents having significant bystander effects can be used, with the effect that the cytotoxic effect of the agent can affect nearby tumor cells to which the antibody may not have bound, even though relatively small amounts of nearby non-tumor tissue may be affected. By contrast, in instances (e.g., leukemias) in which tumor cells are known or believed to exist as free-living cells or widely and intimately intermixed with non-tumor cells, it is appropriate to select a cytotoxic agent more likely to have an effect substantially only upon a cell to which the agent is bound. Selection and conjugation of cytotoxic agents for use with the antibodies described herein is within the ken of a skilled artisan in this field.

The antibodies described herein can thus be used in a method of killing malignant human cells, the method comprising contacting the cells with an immunospecific portion of monoclonal antibody MAbH11B2C2 having a cytotoxic agent linked thereto. The portion binds with the malignant cells and the cytotoxic agent kills, injures, or inhibits growth or proliferation of the malignant cells.

Cell Manipulation

The tumor cell-binding specificity of MAbH11B2C2 (and immunospecific portions thereof) can be used to physically manipulate human tumor cells, especially malignant tumor cells. For example, the antibody can be used to bind such cells to a substrate or to one another. Further by way of example, association or non-association of detectably-labeled antibody with a cell can be used as a criterion for handling the cell. These properties render the antibody useful in conjunction with a wide variety of known cell sorting, selection, and separation methods.

In one aspect, the antibody is bound with a substrate (e.g., a slab or pellet of a gelatinous material such as polyacrylamide or agarose or a sheet of glass or polymer). A suspension of cells that includes tumor cells is brought into fluid contact with the substrate, permitting binding between the antibody and any cells that display H11B2C2 on their surface. Such cells bind with the antibody, thereby becoming bound with the substrate. Cells that are not bound with the substrate are rinsed, for example with an excess of water, buffer, or a cell culture medium. In the absence of non-specific binding between cells and the substrate, substantially only tumor cells will be bound with the substrate. If desired, the number or type of cells bound with the substrate can be assessed. The cells can be recovered from the substrate (e.g., by rinsing it with a suspension containing HA oligomers), or the substrate can simply be discarded.

Such methods can be used, for example, to remove malignant cells from a body fluid of a patient known or believed to harbor tumor cells. By way of example, blood from a patient can be circulated through a cartridge containing a matrix having the antibody bound thereto. Malignant cells in the patient's blood bind with the antibody and thence to the cartridge. After a selected period of time, the cartridge can be disconnected from the patient's blood circulation. In this way, a disposable cartridge (operated in vitro or implanted for limited in vivo operation) can be used to remove malignant cells from a patient's blood (e.g., following surgical tumor resection). Especially in situations in which malignant or other tumor cells are expected to occur repeatedly in the same patient, an ex vivo system connectable to a patient's blood circulation can be operated repeatedly (e.g., daily, weekly, or monthly) with the same patient. Such systems are, of course, not limited to blood, and can be used in connection with any naturally-occurring body fluid (e.g., peritoneal fluid) or with an introduced fluid (e.g., a peritoneal lavage fluid introduced into a patient specifically for performance of these methods.

Likewise, such methods can be used to capture tumor cells from a fluid obtained from a patient whose tumor status is unknown. In such methods, a fluid from a patient is contacted with a substrate having the antibody bound thereto. Cells which express H11B2C2 on their surface bind with the substrate. After the fluid has been contacted with the substrate, the substrate is examined for the presence of cells bound thereto. If any cells are discovered to be bound with the substrate, their number, cytology, and apparent tissue of origin can be assessed. If desired, the cells can be recovered from the substrate, such as by gross dissection of the substrate or by more specific cell-selection methods, such as laser-capture microdissection of a surface of the substrate. Further tests (e.g., antibody-binding or nucleic acid sequencing) can be performed using the recovered cells.

MAbH11B2C2 (or any construct including two or more immunospecifically-binding regions thereof, such as an F(ab′)2 fragment) can be used to agglutinate cells that exhibit H11B2C2 on their surface or to otherwise bind them to one another. In one embodiment, MAbH11B2C2 itself (which is a divalent IgG molecule) is combined with cells bearing H11B2C2. At least some of the divalent antibody molecules will bind to H11B2C2 epitopes on different cells, thereby binding the cells together. The stoichiometry of antibody-based agglutination reactions are well known and can be consulted to determine an appropriate amount of divalent antibody to use under the circumstances in which agglutination is desired.

Detectably labeled MAbH11B2C2 (and immunospecific portions thereof) can be used to differentiate tumor cells from non-tumor cells (and, more specifically, malignant cells from non-malignant cells) in cell-sorting methods, such as various flow cytometric methods.

In one embodiment of such methods, cells in a liquid suspension are assessed for a characteristic and sorted into two or more populations, depending on presence, absence, or intensity of the characteristic in individual cells or in clumps of multiple cells. In the methods described herein, the relevant characteristic is expression of H11B2C2. To sort cells in a suspension based on their expression of H11B2C2, the cells are contacted with the antibody, the antibody is associated with a detectable label (e.g., a fluorophore), and cells or clumps are assessed (e.g., by fluorescent spectroscopic analysis of individual cells or clumps in a flowing, divertable stream of liquid) for the presence of the detectable label bound thereto. Cells or clumps having the label bound thereto are separated from cells or lumps not having the label associated therewith. If desired, cells or clumps having the label associated therewith can be separated into sub-populations, based on the intensity or amount of label detected in association therewith. Thus, a suspension of cells can be separated into populations of non-tumor cells (which do not have an elevated amount of label associated therewith relative to normal cells of the same type, malignant tumor cells (which have the label associated therewith at a relatively higher intensity or density than non-malignant cells of the same type). In addition cells having the label associated therewith can be segregated into various groups, such as cells which exhibit label binding at relatively higher and lower levels, or otherwise according to the extent of antibody binding.

Such suspended-cell sorting techniques can be performed according to substantially any known flow cytometric techniques, and a wide variety of such techniques are known. Selection of an appropriate label for use in connection with such techniques, as well as appropriate equipment and reagents is within the ken of a skilled artisan in this field. The antibody and fragments described herein can be used in combination with other known reagents for specifically labeling cells, and such multi-label methods are well known in the field of flow cytometry.

In another embodiment of such methods, cells on a surface or within a solid or semi-solid tissue sample are labeled with MAbH11B2C2 (or an immunospecific portion thereof) having a detectable label associated therewith. Labeled cells (or portions of a sample with which the antibody specifically binds) are separated from other cells (or portions) that are not substantially labeled, either through gross dissection methods (e.g., manually cutting a sample using a scalpel or breaking a substrate along pre-scored boundaries) or through microscopic methods (e.g., selecting individual cells for collection using laser capture microdissection). As with suspended cell samples, one or more other staining or labeling reagents can be used to permit selection of cells or clumps of cells based on multiple criteria. Using such methods, individual cells or tissue regions can be selected for further analysis. Furthermore, when the sample is a solid tissue sample (e.g., a mounted tissue sample slice), the relative geometry (e.g., location near or within identifiable tissue features) and intensity of antibody staining can be used as additional criteria for selecting cells.

H11B2C2 Protein

MAbH11B2C2 described herein binds immunospecifically with a human protein having a molecular size, as assessed by SDS-PAGE, of approximately 57 kDa, and this protein is herein designated H11B2C2. H11B2C2 binds with HA, and binding between HA and H11B2C2 is inhibited by monoclonal antibody MAbH11B2C2.

Its ability to bind HA indicates that H11B2C2 is a protein that is involved with interaction between cells that express H11B2C2 on their surface and HA-containing ECMs. Expression of H11B2C2 on tumor cells, and more especially on malignant tumor cells indicates that H11B2C2 is involved in interactions of tumor cells, including malignant tumor cells with HA and HA-containing ECMs. Its ability to bind HA intracellularly, including HA associated with chromosomes and other nuclear components, indicates that H11B2C2 is also a protein involved in regulation of gene expression.

Protein H11B2C2 can be isolated by any of a variety of well known affinity chromatography techniques, and monoclonal antibody MAbH11B2C2 can be used as the affinity component in such methods. The protein has also been observed to exist in a form having a molecular size, as assessed by SDS-PAGE, of approximately 37 kDa, and this form is also immunospecifically bound by monoclonal antibody MAbH11B2C2. Without being bound by any particular theory of operation, the 37 kDa form of H11B2C2 is believed to be either a cleavage product of the 57 kDa form or an alternatively spliced version of H11B2C2 that preserves the epitope to which MAbH11B2C2 binds.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

### Example 1

Previous work referring to HABP H11B2C2 was published at Kumarswamy et al., 2008 Adv. Biol. Res. 2(1-2):6-12. However, the Kumarswamy publication did not disclose the identity of H11B2C2, MAbH11B2C2, or how to make either of these. That shortcoming has been overcome by deposit of hybridoma HybH11B2C2 at ______ on ______ (deposit No. ______). Methods of making MAbH11B2C2 using the deposited hybridoma are known to skilled artisans in this field. The disclosure in the Kumarswamy publication, taken together with the availability of MAbH11B2C2 enabled herein and by the corresponding deposit of HybH11B2C2, can be used to isolate H11B2C2 from any tissue in which it is expressed, including any of the malignant tumor tissues described herein. By way of example, H11B2C2 protein can be isolated by passing an extract of cells or tissues which express H11B2C2 (e.g., malignant breast tumor samples) through an affinity chromatography having MAbH11B2C2 bound therewith and collecting aliquots of the effluent from the column as of buffer including increasing salt concentration is passed through the column. Affinity column chromatography methods are well known.

### Example 2

Identification of H11B2C2 Protein and Its Expression in Human Malignant Tumors

The materials and methods employed in the experiments described in this example are now described.

Assay of Human H11B2C2 Protein Expression Using Monoclonal Antibody MAbH11B2C2

Partial purification of H11B2C2 protein from cancer cell lines was achieved by ion exchange chromatography. In the partial purification method, a 5 milliliters (bed volume) DEAE-cellulose column was equilibrated with 50 millimolar sodium acetate buffer (pH 6). Protein extracted from the cell lines was passed through the column. Eluent was collected and the column was washed with four 10 milliliter aliquots of the same buffer. Protein was eluted by stepwise application to the column of the same buffer containing increasing concentrations of NaCl, 0.15 molar, 0.25 molar, 0.4 molar, and 0.7 molar. Fractions having a volume of 1 milliliter each were collected and protein content was measured by spectroscopy at 280 nanometer wavelength. Fractions that exhibited maximum absorbance and amounts of H11B2C2 protein as assessed by SDS-PAGE were used for the ELISA and dot blot experiments.

Reactivity of human carcinoma cell lines with MAbH11B2C2 was assessed using ELISA and dot blot immunoassay formats.

For the ELISA format, semi-purified H11B2C2 (2.5 microgram total protein per milliliter) derived from colon and breast carcinoma cell lines was adsorbed in the wells of 96-well plates at 4 degrees Celsius in presence of sodium azide. The wells were washed with phosphate buffered saline (PBS) containing 0.1% (w/v) bovine serum albumin, 0.1% (w/v) human serum, and 0.1% (v/v) Tween. A solution containing a 1:100 dilution of MAbH11B2C2 was added to each well and incubated for 16 hours at 4 degrees Celsius. The wells were washed with PBS containing 0.1% (w/v) bovine serum albumin and 0.1% (v/v) Tween. Biotinylated goat anti-mouse IgG was added to the wells, which were rinsed prior to receiving a suspension containing streptavidin peroxidase. Color was developed using a colorimetric peroxidase reagent (tetramethylbenzidine/H₂O₂ or 2,2′-Azino-bis(3-Ethylbenzthiazoline-6-Sulfonic Acid)).

For the dot blot assay, H11B2C2 (an amount of the partially purified mixture containing 1.0 microgram total protein) was adsorbed onto nitrocellulose (onto an area of about 0.25-0.5 square centimeters), which was then soaked in solution containing 0.1% (v/v) of each of bovine serum and human serum. The treated membrane was contacted with a solution containing MAbH11B2C2 at various concentrations (1:100 to 1:1000 dilution) for 60 minutes at room temperature, and then washed with a solution consisting of 50 millimolar Tris buffer, 0.15 molar NaCl, and 0.1% (v/v) Tween at pH 8.0. The washed membrane was reacted with biotinylated goat anti-mouse IgG (diluted 1:250 over the commercially obtained stock preparation), rinsed, and contacted with a solution containing streptavidin peroxidase (diluted 1:500 over the commercially obtained stock preparation). Color was developed using DAB (3.3′-diaminobenzidine) reagent. For dot blot assays to assess specificity of HA-binding, the same procedure was used, except that after treating the membrane with H11B2C2, the membrane was incubated overnight in the presence or absence of 500 microgram vitreous HA or with HA oligosaccharides, before being reacted with the biotinylated goat anti-mouse IgG.

Immunohistochemistry

Carcinoma cell line cultures were grown on cover slips for 3 to 4 days, rinsed with PBS, and fixed for 60 seconds in a solution consisting of a 1:1 ratio of methanol:acetone that had been pre-chilled to 4 degrees Celsius. Each fixed sample was dried and rinsed with PBS, and treated with a solution containing 0.1% (v/v) of each of bovine and human serums for 30 minutes at room temperature. The treated cover slips were rinsed with PBS and contacted with a 1:100 dilution of MAbH11B2C2 for 16 hours at 4 degrees Celsius in a humidified chamber. The cover slips were then rinsed with PBS containing 0.1% (v/v) Tween and contacted with a suspension of biotinylated goat anti-mouse IgG for 60 minutes at room temperature. The slips were again rinsed with PBS containing 0.1% (v/v) Tween, contacted with a suspension containing streptavidin peroxidase, and color was developed using DAB.

Biopsied tissue samples were fixed in 10.0% (v/v) buffered formalin and generally included both tumor tissues and adjacent normal tissues. Sections of the tissue sample were deparaffinized using xylene, rehydrated in PBS or in an acetate buffer (pH 5.0) containing 0.15 M NaCl. Substantially identical sections were stained using hematoxylin and eosin stain for the purpose of histopathological screening, and quenched using PBS containing 0.2 M glycine. Endogenous peroxidase activity was quenched by incubating samples in methanol containing 3.0% (v/v) hydrogen peroxide for 30 minutes at room temperature.

Tissue samples were contacted with a solution including 10% (v/v) human serum, 10.0% (v/v) non-fat milk for 60 minutes. Sections were then rinsed with PBS containing 0.1% (v/v) Tween and incubated with MAbH11B2C2 at a 1:10 dilution in PBS containing 0.1% (v/v) human serum for 16 hours at 4 degrees Celsius. The samples were rinsed with PBS containing 0.1% (v/v) Tween, and then incubated with biotinylated goat anti-mouse IgG (diluted 1:250 over the commercially obtained stock preparation) for 60 minutes. These samples were rinsed with PBS containing 0.1% (v/v) Tween and then incubated with a suspension of streptavidin peroxidase (diluted 1:500 over the commercially obtained stock preparation) in a solution of Tris buffer and 0.1% (v/v) human serum for 60 minutes. Color was developed using DAB reagent. Colored samples were soaked in distilled water and dehydrated through graded alcohol solutions, and excess color was cleared in a mixture of isopropanol, acetone and xylene. Slides were mounted in DPX mounting media to preserve the specimen and aid in microscopic imaging (SRL Company, India).

The study population consisted of 202 tissue samples collected from various types of benign and malignant tumor specimens obtained from hospitals during 1998 to 2005. All tissues were fixed in 10% buffered formalin, embedded in paraffin, and sectioned into three- to five-micrometer thick slices. In each case, diagnosis (i.e., malignant tumor, non-malignant tumor, or normal tissue) was made by three independent pathologists through routine hematoxylin and eosin (H&E) staining. Each malignant tumor was graded using the TNM grading system. A total of eight different types of benign tumors, nine well-differentiated tumors, and fifteen poorly differentiated tumors were observed in the samples. For a few tumors, only 3 or 4 tissues samples for the same type of tumor could be obtained. However, for most tumor types we obtained more than 5 tissue samples for the same type of tumor, as detailed in Table I. Therefore, a total number of 200 benign tumor tissue sections, 320 well-differentiated tumor tissue sections, and 450 poorly differentiated tumor tissue sections were prepared and stained for the HAPB expression. In most cases, tissue sections with discrete benign and malignant areas were used based on recommendations made by pathologists. For the present study, well-differentiated tumors were designated grade I, moderately-differentiated tumors were designated grade II, and poorly-differentiated tumors were designated grade III.

In an alternate protocol, frozen tissue sections, a fine needle aspirate (FNAC), or smears containing cells are incubated with MAbH11B2C2 at 4 degrees Celsius and are contacted with biotinylated secondary antibody directed against the MAbH11B2C2, and then with streptavidin, after removing non-specifically-bound MAbH11B2C2 from the sample. The pattern and level of expression of H11B2C2 can be assessed by observing the staining pattern and intensity of fluorescence microscopically.

Western Blot Analysis of Human Tumor Tissues Using MabH11B2C2

Fresh tissue samples were obtained from hospital patients having malignant or benign tumors. Collected tissue samples were maintained in cold PBS-buffer at pH 7.4. After rinsing with the PBS buffer, the tissue samples were extracted by homogenizing them in a lysis buffer (consisting of 50 millimolar Tris buffer at pH 8.0, 20 millimolar EDTA, and 0.5% (v/v) Triton X-100) containing protease inhibitors PMSF (1 millimolar), benzamidine hydrochloride (5 millimolar), and aprotinin (2 millimolar; all obtained from Sigma Chemical Company, St. Louis, USA). The homogenizer used was a Dounce-type glass-glass homogenizer (manufactured by Bellco Glass, Inc. and obtained from Fisher Scientific), which was operated for approximately 10 minutes to rupture substantially all cells, and samples were maintained on ice (approximately 0 degrees Celsius) during homogenization.

Extracts obtained from homogenized tissue samples were centrifuged at 10,000 rpm (approximately 12,000×g) for 20 minutes, the supernatant was separated from the pellet, and the total protein concentration in the supernatant was measured using the Bradford method (Bradford, 1976, Anal. Biochem. 72:248-254).

For SDS-PAGE, supernatant samples were combined with SDS-PAGE reducing sample buffer to approximately equal protein concentrations, and the diluted samples were boiled. For immunoblot experiments, a total of 100 micrograms of protein obtained from individual cell lysates or tissue extracts was separated by SDS-PAGE on a 10 or 12% (w/v) gel, and the separated proteins were transferred onto a polyvinylidene difluoride (PVDF) membrane (Millipore, Germany). The PVDF membrane was treated with a solution containing 1% (w/v) non-fat dry milk, 1% (w/v) BSA (Sigma Chemical Company, St. Louis, USA) and 10% (v/v) inactivated pathogen-free human serum in TTBS buffer (Tris-buffered saline containing 0.1% Tween) for 1 hour to inhibit non-specific antibody binding to the membrane. Following this treatment, the membrane was contacted with a 1:500 dilution of MAbH11B2C2 for 16 hours at 4 degrees Celsius. After antibody treatment, the membrane was contacted with a 1:5000 dilution of biotinylated goat anti-mouse IgG (H+L) (Sigma Chemical Company, St. Louis, USA) for 1 hour and then with a 1:10,000 dilution of streptavidin-peroxidase (Sigma, St. Louis U.S.A) for 1 hour in TTBS buffer. After each step, the membrane was washed five times with TTBS buffer for 5 minutes for each wash. The antibody and antigen reaction on the immunoblot was visualized using the DAB chromogen substrate (Sigma Chemical Company, St. Louis, USA) or using the ECL reagent (Pierce Chemical Company, Rockford, Ill., USA).

The results obtained from the experiments described in this example are now described.

Detection of MAbH11B2C2-reactive HABP in in Vitro Cultured Cells

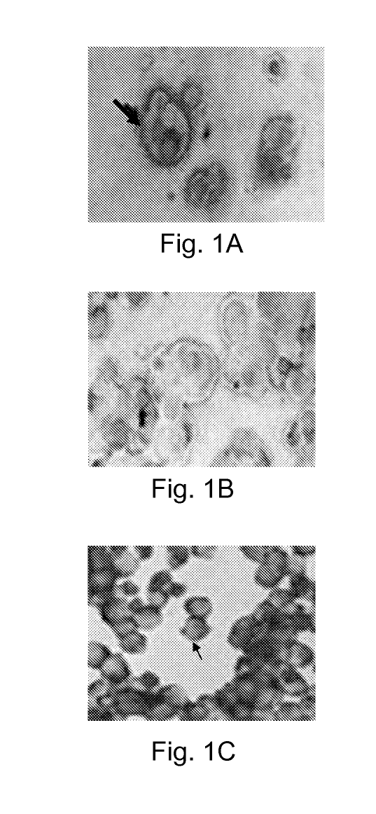

Immunocytochemical studies of cultured human cancer cells demonstrated over-expression of MAbH11B2C2-reactive HABP. HABP expression was observed both on the nuclear surface and the cell surface, as illustrated in FIG. 1.

Breast cancer cell line-MDA MB436 exhibited reactivity with MAbH11B2C2. Cells reacted with this antibody and stained exhibited accumulation of H11B2C2 protein on the cell membrane and at the nuclear surface, as shown in FIG. 1A.

Colon cancer cell line-320 DM exhibited reactivity with MAbH11B2C2. Cells reacted with this antibody and stained demonstrated the presence of H11B2C2 protein at the cell surface and at the nucleus, as shown in FIG. 1B.

Prostate cancer cell line-PC 3 exhibited reactivity with MAbH11B2C2. Cells reacted with this antibody and stained exhibited expression of H11B2C2 all over the cells, as shown in FIG. 1C.

Immunohistochemical Analysis of Human Tumor Samples Using MAbH11B2C2

Expression of H11B2C2 by hyperplastic, benign tumor, and malignant (invasive stage 2 or 3) tumor samples was assessed by staining using MAbH11B2C2. The specificity of the antibody interaction was confirmed by competition study in the presence of at 500 micrograms per milligram of HA-oligosaccharides. With the exception of in situ carcinoma of uterine cervix samples (obtained from Papanicolaou {Pap} smears), samples were derived from formalin-fixed, paraffin-embedded specimens. Tumors were diagnosed initially by routine hematoxylin and eosin (H&E) staining The results obtained using various tissue samples are discussed below, and representative examples of those results are depicted in the Figures and summarized in Table I.

Most samples included both malignant and adjacent non-malignant tissue areas (i.e., in the same tissue obtained from same patient). Samples also included malignant and benign tumors (for the same type of tissue) from multiple individuals for most tumor types. Table I indicates the number of malignant and benign tumor samples that were used for the experiments described herein.

A simple scoring method was used for—semi quantitative analysis of HABP staining in benign and malignant tumor tissue sections (as assessed using MAbH11B2C2). This method was primarily based on visual interpretation of staining intensity independently by three individuals. HABP staining intensity was scored on a scale of 1-4, wherein 1 denotes weak staining, 2 denotes moderate staining, 3 denotes strong staining, and 4 denotes maximum staining. The same scoring method was used for visual interpretation when comparing HABP staining intensity of tumor cells and HABP staining intensity of surrounding stroma in the benign and malignant tumor tissue sections. This scoring system is the one used in Table I. Any discrepancies in the scores were resolved with further discussion among the scoring individuals. Correlation between the staining intensity and tumor grades was analyzed using the non-parametric Spearman's rank correlation method. Comparisons of HABP staining specificity between tumor cells and surrounding stroma was analyzed using the Wilcoxon signed-rank test. Statistical analysis was performed by using the PRISM 4 (™, GraphPad Software, Inc) brand statistical analysis software package and a value of p<0.05 was considered statistically significant.

TABLE I

| | Number of Tissue | Staining Intensity Score | |
|---|---|---|---|
| Tumor Tissue | Samples Observed | Tumor | Stroma |

BENIGN TUMORS

| | | | |
|---|---|---|---|
| Benign Parotid Tumor | 3 | 2 | Stroma absent |
| Benign Breast Tumor | 17 | 1.75 | 1 |
| Benign Stomach Tumor | 12 | 1.75 | 1 |
| Benign Pancreatic Tumor | 3 | 1 | 1 |
| Benign Tumor of Descending Colon | 6 | 1 | 1 |
| Benign Rectal Tumor | 5 | 1 | 1 |
| Benign Prostate Tumor | 5 | 1 | 1 |
| Benign Kidney Tumor | 3 | 1 | 1 |

WELL-DIFFERENTIATED TUMORS

| | | | |
|---|---|---|---|
| Parotid Mucoepidermoid Carcinoma | 5 | 2 | Stroma absent |
| Thyroid Papillary Carcinoma | 4 | 2 | 1 |
| Breast Infiltrating Ductal Carcinoma | 12 | 2.5 | 1 |
| Stomach Adenocarcinoma | 5 | 2.5 | 1 |
| Pancreatic Adenocarcinoma | 5 | 2.5 | 1 |
| Adenocarcinoma of Descending Colon | 5 | 2 | 1 |
| Transitional Cell Carcinoma of | 6 | 2.5 | 1 |
| Urinary Bladder | | | |
| Prostate Adenocarcinoma | 7 | 2 | 1 |
| Non-Hodkin's Lymphoma | 4 | 2 | 1 |

POORLY-DIFFERENTIATED TUMORS

| | | | |
|---|---|---|---|
| Ameloblastoma | 2 | 4 | 1 |
| Parotid Mucoepidermoid Carcinoma | 3 | 4 | 2 |
| Breast Medullary Carcinoma | 4 | 4 | Stroma absent |
| Stomach Adenocarcinoma | 21 | 4 | 1 |
| Gall Bladder Adenocarcinoma | 6 | 4 | 1 |
| Pancreatic Adenocarcinoma | 8 | 4 | 1 |
| Adenocarcinoma of Descending Colon | 4 | 4 | 1.25 |
| Rectal Adenocarcinoma | 6 | 3 | 1.25 |
| Transitional Cell Carcinoma of | 4 | 4 | 1 |
| Urinary Bladder | | | |
| Ovarian Serous Cyst Adenocarcinoma | 7 | 4 | Stroma scanty |
| Testicular Seminoma | 6 | 3 | Stroma absent |
| Prostate Adenocarcinoma | 10 | 4 | 1.75 |
| Osteosarcoma | 4 | 4 | 1.75 |
| Wilms' Tumor | 2 | 4 | 1.75 |
| Medulloblastoma | 8 | 4 | Stroma scanty |

Thyroid: Papillary Carcinomas

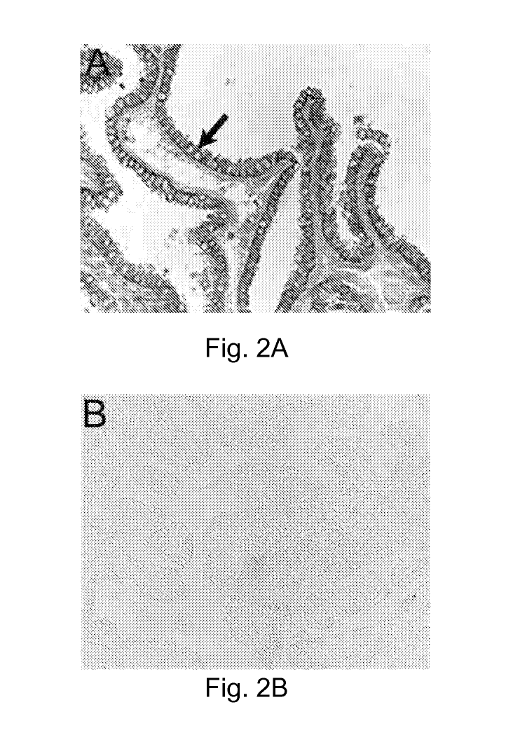

Papillary carcinoma of thyroid was used for present study. Cells lining colloidal spaces forming papillary structures were highly reactive to MAbH11B2C2. These results demonstrate that H11B2C2 is over-expressed both at the basal surfaces and surrounding the adjacent cells (see FIG. 2A) of malignant papillary carcinoma of thyroid. The MAbH11B2C2-reactivity was significantly reduced (as shown in FIG. 2B) in a competition study in which 250 micrograms per milliliter of HA was present, confirming the specificity of the antibody-antigen reaction. No staining was observed for tumor sections when MAbH11B2C2 was omitted from the staining procedure.

Parotid: Mixed Parotid Tumors

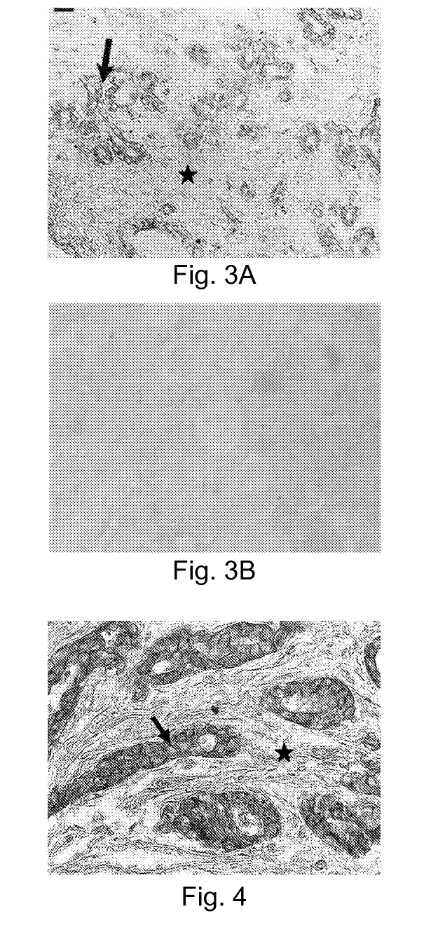

These tumors had both squamous and adenocarcinomatous components. Adjacent benign areas with typical ductal structures showed moderate reactivity to MAbH11B2C2, while both squamous and adenocarcinomatous (malignant) cell surfaces exhibited strong reactivity to MAbH11B2C2 antibody in extra and inter cellular spaces, demonstrating over-expression of H11B2C2, as shown in the region of FIG. 3A designated by an arrow. On the other hand, stroma with fibrous tissues showed almost no MAbH11B2C2 reactivity (star in FIG. 3A). In a control experiment, non-specific antibody H3 exhibited no reactivity (FIG. 3B).

Breast: Infiltrating Duct and Medullary Breast Carcinomas

Immunostaining of both infiltrating ductal and medullary malignant carcinomas using MAbH11B2C2 demonstrated over-expression of H11B2C2 by these cells. Migrating tumor cords infiltrated through the stroma showed over-expressed H11B2C2, both at the cell surfaces and between the cells (areas indicated by the arrow in FIG. 4). Stromal areas showed negligible H11B2C2 expression (star in FIG. 4). Infiltrating lymphocytes were also highly reactive to MAbH11B2C2 antibody. No reactivity was observed either when the samples were contacted with MAbH11B2C2 in the presence of 250 micrograms per milliliter of HA or when the samples were contacted with non-specific antibody H3.

Stomach: Stomach Adenocarcinomas

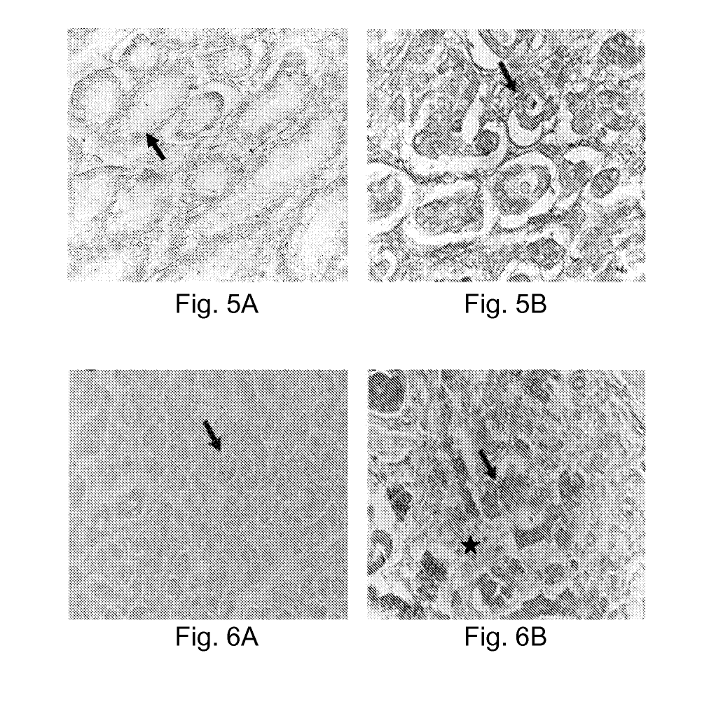

Hyperplastic tissue regions exhibited limited, specific regions of reactivity with MAbH11B2C2, but most tissue areas were non-reactive (see FIG. 5A). In a sample including malignant tumor regions, tumor cells spread in a disorderly manner in islands and in small clusters, and these islands and clusters exhibited strong reactivity with MAbH11B2C2 (see FIG. 5B). Tissues treated with hyaluronidase to unmask cryptic sites of H11B2C2 expression, showed moderate to high reactivity with MAbH11B2C2. No staining was observed for tumor sections when MAbH11B2C2 was omitted from the staining procedure.

Pancreas: Pancreatic Adenocarcinomas

Normal pancreas tissue area having typical acinar structure were non-reactive with MAbH11B2C2 (see FIG. 6A). Benign acinar cells exhibited moderate to no reactivity with MAbH11B2C2. In tissue sections including malignant tumors, tumors were scattered in aggregates throughout the stroma. Migrating tumors were strongly reactive with MAbH11B2C2 (see FIG. 6B). No staining was observed for tumor sections when MAbH11B2C2 was omitted from the staining procedure, indicating that H11B2C2 expression was limited to tumor tissues only.

Gall Bladder: Adenocarcinomas

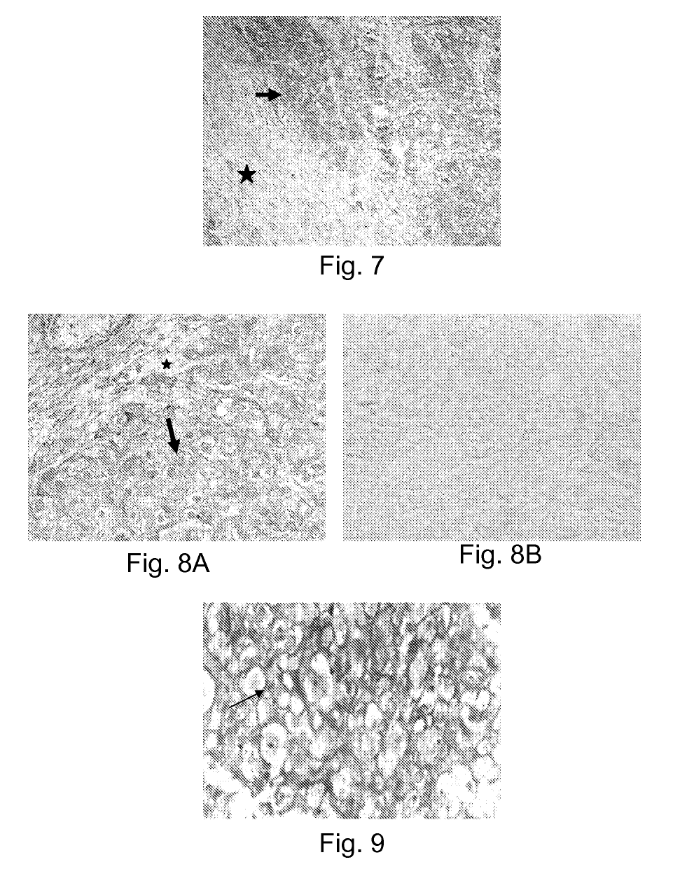

Aggregated and firm tumor cells exhibited strong reactivity with MAbH11B2C2. Negligible reactivity with MAbH11B2C2 was observed in the stromal regions (see FIG. 7). No staining was observed for tumor sections when MAbH11B2C2 was omitted from the staining procedure.

Urinary Bladder: Transitional Cell Carcinomas

Both papillary and solid tumors exhibited high expression of H11B2C2. In malignant tumor tissue sections, strong staining was observed both on the cell membrane and in the nucleus when reacted with MAbH11B2C2 (see FIG. 8A). Treatment of samples with hyaluronidase revealed additional cryptic H11B2C2 staining on the nuclear regions. Benign areas of the tumors showed negligible reactivity with MAbH11B2C2. No staining was observed for tumor sections when MAbH11B2C2 was omitted from the staining procedure. (FIG. 8B).

Colon: Adenocarcinomas

The malignant tumor was moderately differentiated and composed of signet-ring cells. Tumor cells were highly reactive with MAbH11B2C2. The tall columnar glands also over-expressed HABP (see FIG. 9). Approximately eighty percent of MAbH11B2C2 reactivity was eliminated by pre-treatment of tissue sections with HA or HA oligomers.

Rectum: Adenocarcinoma

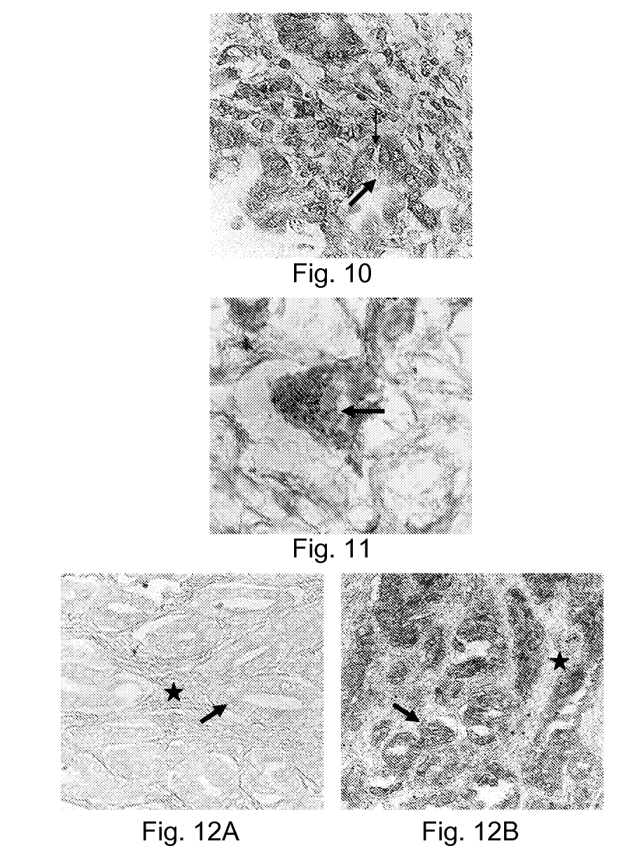

A malignant papillary carcinoma sample exhibiting columnar epithelial cell membranes was strongly reactive with MAbH11B2C2. The stroma and affiliated lymphocytes exhibited moderate reactivity. Tumors in colloidal areas were strongly reactive with MAbH11B2C2 (see FIG. 10).

Cecum: Adenocarcinomas

Malignant colloidal carcinomas exhibited strong reactivity with MAbH11B2C2. Stromal areas exhibited only moderate reactivity. Papillary foldings with columnar cell membrane and associated extracellular spaces exhibited strong reactivity (see FIG. 11). Normal epithelial areas showed no reactivity. Tumor tissues pre-treated with hyaluronidase exhibited more intense reactivity, indicating cryptic sites of HABP expression.

Prostate: Adenocarcinomas

Benign areas of prostate tissue samples containing adenocarcinomas exhibited very low expression of H11B2C2 when stained using MAbH11B2C2 (FIG. 12A). Areas of prostate tissue slices including malignant tumor cells (as assessed microscopically by H&E staining) exhibited significantly higher expression of H11B2C2 (as assessed by staining using antibody MAbH11B2C2), compared with benign areas (FIG. 12B). Pre-treatment of tissue sections with HA substantially eliminated (>90%) staining by MAbH11B2C2.

Ovary: Serous Cystoadenocarcinomas

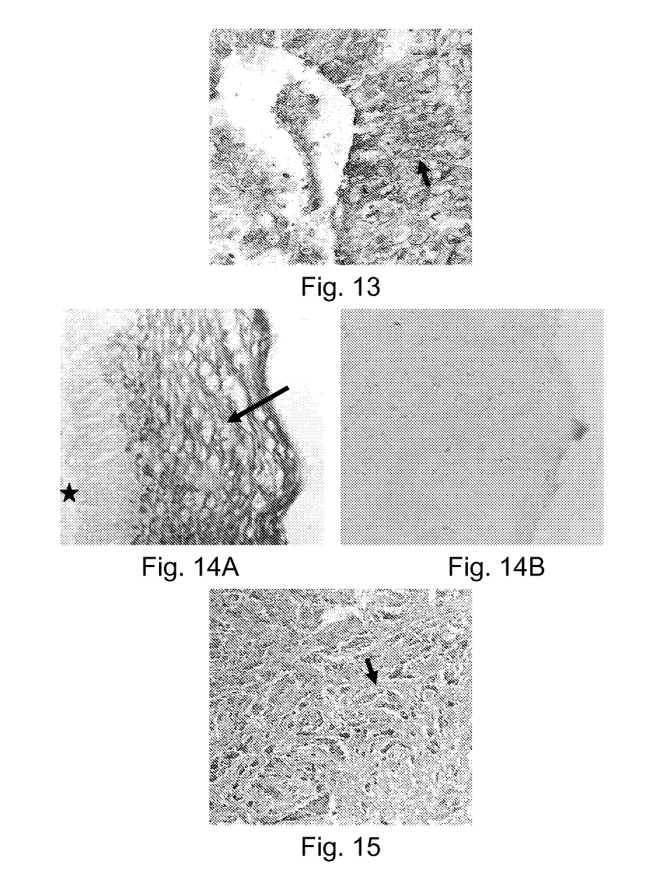

Cells of a malignant serous cystoadenocarcinoma tumor of the right ovary of a patient exhibited significantly greater expression of H11B2C2 than did corresponding non-tumor tissue, as assessed by staining using antibody MAbH11B2C2. In the stained tumor sample, significant expression of H11B2C2 was observed both on the cell surfaces and in the nuclear region (FIG. 13).

Uterine Cervix: Squamous Cell Carcinoma

Cervical tissue sections were stained using MAbH11B2C2. Strong reaction (i.e., heavy staining) was observed at the squamous cell layer and in basal cells that appeared to have lost polarity. Increasing levels of H11B2C2 were detected in cells approaching stromal areas, suggesting that cells express (or accumulate) increasing amounts of H11B2C2 as they migrate toward stromal areas. However, the stromal region showed no reaction with MAbH11B2C2 (FIG. 14A). No staining was observed for tumor sections when MAbH11B2C2 was omitted from the staining procedure (FIG. 14B).

Cervical Smears

150 Cervical cancer and 45 normal cervical smears were fixed using a methanol:acetone mixture, air-dried, and stained using MAbH11B2C2 to detect H11B2C2 expression. These samples were observed to contain stratified squamous epithelia with distinct nuclei surrounded with fibrous tissues and blood. All cancer smears exhibited expression of H11B2C2 at significantly greater levels than corresponding non-cancerous smears, as assessed by staining with MAbH11B2C2. Normal cervical smears showed no reaction in their epithelia, in their nuclei, or in the extracellular spaces.

Brain: Medulloblastomas

Medulloblastoma samples were determined to be infiltrative, undifferentiated cellular neoplasms by H&E staining Tumor cells were atypical, carrot-shaped cells having no visible cytoplasm. Some areas of tumors were infiltrated with fibrillated processes. The carrot-shaped tumor cells expressed H11B2C2 at significantly greater levels than corresponding non-tumor tissues, as assessed by staining with MAbH11B2C2. Moderate staining with this antibody was observed in tissue surrounding the tumors (FIG. 15).

Kidney: Nephroblastomas (Wilms' Tumor)

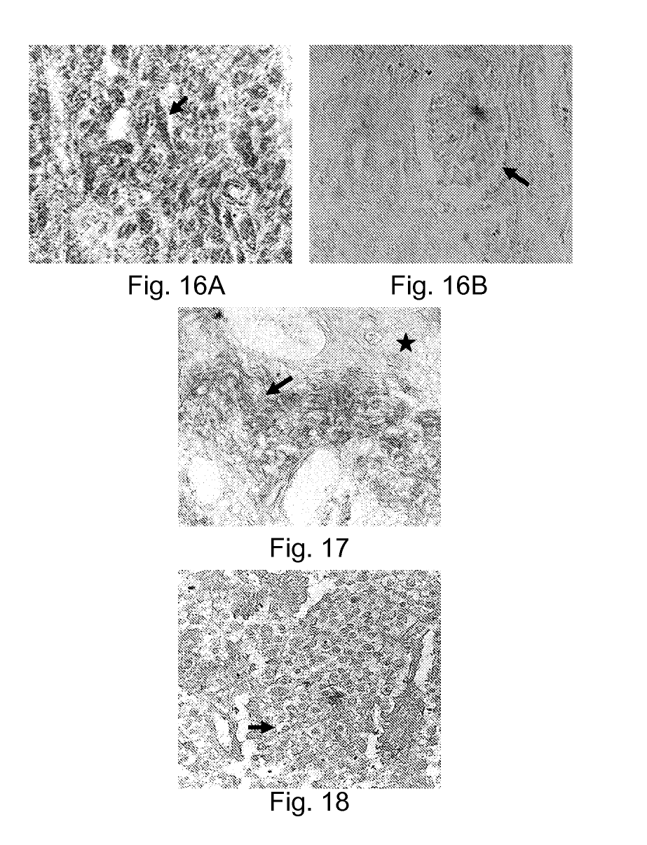

These tumors exhibited a lack of cytoplasm and hyperchromatic nuclei. Some tumor areas showed normal glomerular structures. Upon staining with MAbH11B2C2, the cord-like malignant tumors were strongly reactive with the antibody indicating over-expression of H11B2C2, relative to non-tumorous tissue (FIG. 16A). Tumor areas exhibiting normal-looking glomerular structure were substantially not stained by MAbH11B2C2 (FIG. 16B). No staining was observed for tumor sections when MAbH11B2C2 was omitted from the staining procedure. Also, pre-treatment of tissue sections with HA oligomers substantially eliminated staining by MAbH11B2C2

Bone: Osteosarcomas

Tissues containing osteosarcomas were derived from leg bones. Tissue sections exhibited extensive tumor migration in the outer peritoneum, as assessed microscopically after H&E staining Osteosarcoma-containing regions of the samples exhibited osteoblasts having little cytoplasm and densely-packed, highly hyperchromatic nuclei. Tumor areas exhibited numerous infiltrating lymphocytes. Spicules of osteoid were lined by neoplastic osteoblasts in these areas. Osteoblasts in tumor areas stained strongly with antibody MAbH11B2C2 (FIG. 17). Intercellular material also exhibited positive reaction with the antibody. Stromal areas exhibited substantially less staining with MAbH11B2C2. No staining was observed for tumor sections when MAbH11B2C2 was omitted from the staining procedure. Also, pre-treatment of tissue sections with HA substantially eliminated staining by MAbH11B2C2.

Testes: Seminomas

These tissue samples contained malignant tumor cells grouped into lobules and surrounded by compressed stromal septa. The tumor lobules contained migrating lymphocytes. Tumor nuclei and outer membranes reacted strongly with antibody MAbH11B2C2, indicating significant expression of H11B2C2 at these cellular locations, relative to non-tumor cells (FIG. 18). Pre-treatment of tissue samples with hyaluronidase prior to MAbH11B2C2 staining did not alter staining of the nuclei. However this pre-treatment resulted in a marked increase in staining of the cell membrane.

Lymphocytes: Non Hodgkin's Lymphomas

Lymphocytes in these tumor samples were highly reactive with MAbH11B2C2 antibody. The peripheral beta cells in particular exhibited increased expression of H11B2C2, relative to non-tumor lymphocytes. Medullary regions were less reactive with the antibody than the peripheral beta cells.

Western Blot Analysis of Human Malignant and Non-Malignant Tissues:

Western blot analyses were performed using proteins extracted from fresh tissues taken from human malignant and benign tumor samples. These proteins were subjected to 10% SDS-PAGE under denaturing condition. Separated proteins were transferred onto PVDF membranes and reacted with MAbH11B2C2. Proteins which bound with MAbH11B2C2 were visualized.

The results of these experiments demonstrated over-expression of a protein having an approximate size of 57 kDa and identified herein as H11B2C2 in malignant tumor tissue, relative to expression levels observed in corresponding benign tissues of the same types. We screened 13 different types of malignant tumor tissues, and the 57 kDa H11B2C2 was expressed in all of them. These tumor types include endometrial cancer, cheek cancer, breast cancers, stomach cancer, cervical cancer, tongue cancer, colon cancer, ovarian cancer, gall bladder cancer, buccal mucosal cancer, duodenal cancer, rectal cancer, and laryngeal cancer.

In addition to the 57 kDa H11B2C2 protein, we also observed limited reactivity of MAbH11B2C2 with a minor protein band having an approximate size of 37 kDa. This 37 kDa protein could be an isomer, cleavage product, or degradation product of the 57 kDa H11B2C2.

Taken together, the experiments and observations discussed in this example demonstrate that over-expression (relative to corresponding non-tumor tissue of the same type) of H11B2C2 protein is diagnostic of malignancy for each of the following tumor types:
brain cancers
tongue cancers
buccal mucosal cancers
parotid cancers
laryngeal cancers
stomach cancers
colon cancers
duodenal cancers
rectal cancers
kidney cancers
bladder cancers
gall bladder cancers
pancreatic cancers
thyroid cancers
breast cancers
prostate cancers
testicular cancers
ovarian cancers
endometrial cancers
cervical cancers
bone cancers
lymphatic cancers.

Although the foregoing list does not include all known types of cancers, it is significant that over-expression of H11B2C2 appears to be diagnostic of every metastatic cancer type that has been tested, including all of those in the foregoing list. These results indicate that over-expression of H11B2C2 can be used to assess the malignant status of substantially any type of tissue.

Furthermore, the data disclosed herein suggest that low levels of over-expression of H11B2C2 can occur in benign tumors, relative to the corresponding non-tumor tissue of the same type. It is possible to estimate the degree of over-expression relative to non-tumor tissue of the same type, that is diagnostic of metastatic tumor occurrence and benign tumor occurrence (see data in Table I).

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

1. An immunospecific portion of a monoclonal antibody that specifically binds with a hyaluronic acid-binding protein expressed by malignant human cells, the monoclonal antibody being MAbH11B2C2.

2. The immunospecific portion of claim 1, being detectably labeled.

3. The immunospecific portion of claim 1, having a cytotoxic agent conjugated therewith.

4. The immunospecific portion of claim 3, wherein the cytotoxic agent is selected from the group consisting of radionuclides, cytotoxins, viruses, and combinations of these.

5. The immunospecific portion of claim 1, being MAbH11B2C2.

6. A method of assessing malignancy of a human cell, the method comprising contacting the cell with an immunospecific portion of monoclonal antibody MAbH11B2C2 and assessing whether the cell and the portion specifically bind, whereby specific binding between the cell and the portion indicates that the cell is a malignant cell.

7. The method of claim 6, wherein the cell is contacted with the portion in vitro.

8. The method of claim 7, wherein the immunospecific portion detectably labeled.

9. The method of claim 8, wherein the cell is adhered to a substrate.

10. The method of claim 7, wherein the immunospecific portion is adhered to a substrate.

11. The method of claim 7 further comprising rinsing the cell, after contacting it with the immunospecific portion, with a fluid to substantially remove any of the immunospecific portion that is not specifically bound with the cell.

12. The method of claim 11, further comprising contacting the cell with a detectable reagent that specifically binds with the immunospecific portion, whereby detection of the reagent associated with the cell indicates that the cell is a malignant cell.

13. The method of claim 12, wherein the detectable reagent is an enzyme that catalyzes a colorigenic reaction.

14. The method of claim 13, wherein the immunospecific portion is biotinylated and the enzyme is linked with an avidin.

15. The method of claim 12, wherein the detectable reagent is a detectably-labeled protein that specifically binds with the immunospecific portion.

16. The method of claim 15, wherein the protein is an antibody that immunospecifically binds with mouse antibodies.

17. The method of claim 12, further comprising rinsing the cell, after contacting it with the reagent, with a fluid to substantially remove any of the reagent that is not associated with the cell.

18. The method of claim 7, wherein specific binding between the cell and the immunospecific portion is assessed by microscopic observation of the cell.

19. The method of claim 6, wherein the cell is selected from the group consisting of brain cells, tongue cells, buccal mucosal cells, parotid cells, laryngeal cells, stomach cells, colon cells, duodenal cells, rectal cells, kidney cells, bladder cells, gall bladder cells, pancreatic cells, thyroid cells, breast cells, prostate cells, testicular cells, ovarian cells, endometrial cells, cervical cells, bone cells, and lymphatic cells.

20. (canceled)

21. A method of assessing malignancy of a human sample cell, the method comprising contacting the sample cell with an immunospecific portion of monoclonal antibody MAbH11B2C2 and comparing:
a) the degree of specific binding between the immunospecific portion and the sample cell and
b) the degree of specific binding between the immunospecific portion and a human control cell, wherein the control cell is a cell of the same type as the sample cell and the control cell is known not to be a malignant tumor cell,
whereby a significantly greater degree of specific binding between the immunospecific portion and the sample cell than between the immunospecific portion and the control cell indicates that the sample cell is a malignant cell.

22-39. (canceled)
